(19) United States
(12) Patent Application Publication
(10) Pub. No.: US 2014/0329714 A1
(43) Pub. Date: Nov. 6, 2014

(54) STAT3 ACTIVATION AS A MARKER FOR CLASSIFICATION AND PROGNOSIS OF DLBCL PATIENTS

(71) Applicant: ALBERT EINSTEIN COLLEGE OF MEDICINE OF YESHIVA UNVIERSITY, Bronx, NY (US)

(72) Inventors: Bihui H. Ye, New Rochelle, NY (US); Wing (John) Chan, Omaha, NE (US); Kai Fu, Omaha, NE (US)

(73) Assignees: ALBERT EINSTEIN COLLEGE OF MEDICINE OF YESHIVA UNIVERSITY, Bronx, NY (US); BOARD OF REGENTS OF THE UNIVERSITY OF NEBRASKA, Lincoln, NE (US)

(21) Appl. No.: 14/360,325

(22) PCT Filed: Nov. 28, 2012
(86) PCT No.: PCT/US12/66782

§ 371 Date: May 23, 2014

Related U.S. Application Data
(60) Provisional application No. 61/564,423, filed on Nov. 29, 2011.

(51) Int. Cl. C12Q 1/68

(52) U.S. Cl. CPC C12Q 1/6886; C12Q 2600/112
USPC 506/9; 506/16

(57) ABSTRACT

Methods are disclosed for determining classification and prognosis of patients with diffuse large B-cell lymphoma (DLBCL) using activation of signal transducer and activator of transcription 3 (STAT3).

### CROSS-REFERENCE TO RELATED APPLICATION

This application claims the benefit of U.S. Provisional Patent Application No. 61/564,423, filed Nov. 29, 2011, the content of which is herein incorporated by reference in its entirety.

### STATEMENT OF GOVERNMENT SUPPORT

This invention was made with government support under grant numbers CA85573 and CA114778 awarded by the National Institutes of Health. The government has certain rights in the invention.

### BACKGROUND OF THE INVENTION

Throughout this application various publications are referred to by superscripts. Full citations for these references may be found at the end of the specification. The disclosures of these publications are hereby incorporated by reference in their entirety into the subject application to more fully describe the art to which the subject invention pertains.

Diffuse large B-cell lymphoma (DLBCL) is the most common lymphoid malignancy in the adult population and accounts for about 40% of newly diagnosed non-Hodgkin lymphoma cases.¹ When treated with anthracyclin-based chemotherapy regimens such as a combination of cyclophosphamide, doxorubicin, vincristine, and prednisone (CHOP), the 5-year overall survival rate of DLBCL is approximately 50%.² Addition of Rituximab to the standard CHOP regimens (R-CHOP) results in an improvement of overall survival rate by 10 to 15%.³ Nevertheless, a substantial number of patients still succumb to the disease and hence improvements in therapy remain a necessary and important task.

DLBCL is a biological and clinical heterogenous disease, which is rooted at least in part in the diversity of its normal cell counterparts.⁴ Based upon their gene expression similarities to either normal germinal center (GC) B cells or activated peripheral blood B cells, DLBCLs can be classified into two main subcategories: germinal center B-cell-like (GCB) DLBCL and activated B-cell-like (ABC) DLBCL.^{5,6} The GCB-DLCBL subtype represents transformed counterpart of normal GC centroblasts as both highly express the GC master regulator BCL6 and lack B cell activation features. In comparison, the ABC-DLBCL subtype likely corresponds to cells arrested at the late GC/pre-plasmablastic stage of maturation,⁶ is characterized by constitutively activated NF-κB and shows activation of Jak/STAT3 signaling.⁷⁻⁹ Signal transducer and activator of transcription 3 (STAT3) activation has been identified as an oncogenic event in multiple malignancies, and in ABC-DLBCL cell lines, inhibition of STAT3 signaling leads to tumor cell apoptosis.⁸⁻⁹

The biological difference between the GCB- and ABC-DLBCL subgroups also transpires to different responses to therapy, with GCB-DLBCL having significantly better overall survival rates when treated with the CHOP regimen.¹⁰ Although the survival outcome of ABC-DLBCL patients has been notably improved with the R-CHOP therapy, the survival difference between ABC- and GCB-DLBCL still persists.¹¹⁻¹⁴ It is thus important to identify novel biomarkers that can risk-stratify the ABC-DLBCL patients in the R-CHOP era in order to guide development of targeted therapy.

Aberrantly activated STAT3 has been shown to be oncogenic in a number of malignancies. In normal cells, STAT3 activation in response to growth factor or cytokine receptor signaling is a transient and tightly controlled process due to rapid activation and self-inactivation cycles.¹⁵ In cancer, constitutive activation of the STAT3 signaling pathway promotes tumor cell growth, survival, angiogenesis, and metastasis.¹⁶ Through inflammatory mediators in the tumor microenvironment, tumor cells with activated STAT3 can evade immune surveillance by inhibiting anti-tumor immune responses.¹⁷ In lymphoid malignancies, a pathogenic role of STAT3 has been shown in multiple myeloma, Hodgkin's lymphoma, anaplastic large T-cell lymphoma, and recently, in ABC-DLBCL.^{8,9,18-21} The STAT3 gene is a direct target of BCL6-mediated transcription repression such that BCL6 positive normal GC B cells and GCB-DLBCLs are largely STAT3-low or negative.⁸ Furthermore, treating cultured ABC-DLCBL cells with specific siRNA against STAT3 or a Jak inhibitor induces cell cycle arrest and apoptosis.^{8,9} Analysis by Lam et al further suggested that, in ABC-DLBCL cells, constitutively activated NF-κB pathway may indirectly activate Jak/STAT3 pathway by upregulating the STAT3-activating cytokine IL-6 and/or IL-10.⁹

The present invention addresses the need, using STAT3 activation, for improved methods that can be used for prognosis and risk-stratified therapy of DLBCL patients.

### SUMMARY OF THE INVENTION

The present invention provides methods of classifying a human patient with diffuse large B-cell lymphoma (DLBCL), the method comprising determining mRNA expression levels of human genes in a DLBCL biopsy specimen from the patient, wherein the genes comprise HSD17B4, RNF149, ZNF805, SLC2A13, RHEB, MT1X, NAT8L, C15orf29, ZNF420, PCNX and SLA, so as to classify the DLBCL patient based on expression levels.

The invention also provides methods for classifying a human patient with diffuse large B-cell lymphoma (DLBCL), the methods comprising determining mRNA expression levels of human genes in a DLBCL biopsy specimen from the patient, wherein the genes comprise Module A genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1, and Module B genes BTLA, C13orf18, CFLAR, EV12A, HIST2H2AA3, IL16, IL2RA and PTGER4; and comparing the expression levels of Module A genes with the expression levels of Module B genes so as to classify the DLBCL patient based on the mRNA expression levels.

The invention also provides methods of determining the prognosis of a diffuse large B-cell lymphoma (DLBCL) patient undergoing treatment with rituximab in combination with cyclophosphamide, doxorubicin, vincristine, and prednisone (R-CHOP), or treatment with rituximab in combination with cyclophosphamide, mitoxantrone, vincristine, and prednisone (R-CNOP), the method comprising determining the level of phospho-Tyr705-STAT3 (PY-STAT3) in a DLBCL biopsy specimen from the patient using immunohistochemistry, wherein PY-STAT3 positivity predicts a poor likelihood of survival in comparison to a patient with PY-STAT3 negativity.

The invention further provides methods of determining the prognosis of a diffuse large B-cell lymphoma (DLBCL) patient undergoing treatment with a combination of cyclophosphamide, doxorubicin, vincristine, and prednisone (CHOP), or with a combination of cyclophosphamide, mitoxantrone, vincristine, and prednisone (CNOP), the method comprising determining the level of phospho-Tyr705-STAT3 (PY-STAT3) and the level of BCL6 in a DLBCL biopsy specimen from the patient using immunohistochemistry, wherein PY-STAT3 positivity and BCL6 negativity predicts a poor likelihood of survival in comparison to a patient who is not PY-STAT3 positive and BCL6 negative.

The invention also provides a gene expression profile that is predictive of activation of signal transducer and activator of transcription 3 (STAT3), wherein the profile comprises expression of a plurality of, or all of, the following genes: MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The invention provides a microarray for classifying a human patient with diffuse large B-cell lymphoma (DLBCL), where the microarray comprises nucleic acid probes for genes HSD17B4, RNF149, ZNF805, SLC2A13, RHEB, MT1X, NAT8L, C15orf29, ZNF420, PCNX and SLA.

The invention also provides a microarray for classifying a human patient with diffuse large B-cell lymphoma (DLBCL), where the microarray comprises nucleic acid probes for genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The invention provides a gene expression-based method for classifying a human patient with diffuse large B-cell lymphoma (DLBCL), where the method comprises using nucleic acid probes for detecting expression of genes HSD17B4, RNF149, ZNF805, SLC2A13, RHEB, MT1X, NAT8L, C15orf29, ZNF420, PCNX and SLA.

The invention also provides a gene expression-based method for classifying a human patient with diffuse large B-cell lymphoma (DLBCL), where the method comprises using nucleic acid probes for detecting expression of genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The invention provides a method of classifying a human patient with diffuse large B-cell lymphoma (DLBCL), the method comprising determining STAT3 mRNA expression level in a DLBCL biopsy specimen from the patient, and comparing the level of STAT3 mRNA expression from the patient with the level of expression of STAT3 mRNA from a cohort of DLBCL patients, wherein a patient with a level of STAT3 mRNA expression that is greater than 1 standard deviation above the mean level of STAT3 mRNA expression in the cohort has a less favorable survival outcome compared to patients having a level of STAT3 mRNA expression that is less than 1 standard deviation below the mean level of STAT3 mRNA expression in the cohort.

### BRIEF DESCRIPTION OF THE DRAWINGS

FIG. 1A-1H. Representative immunohistochemical staining of one GCB-DLBCL case (A-D) and one ABC-DLBCL case (E-H). (A) & (E) H&E staining, (B) & (F) PY-STAT3 and CD20 double staining, (C) & (G) BCL6 staining, and (D) & (H) MUM1 staining.

FIG. 2A-2B. Effect of STAT3 siRNA treatment on the endogenous STAT3 protein levels (A) and caspase activation (B). DLBCL cells were transiently transfected using Nucleofector Kit T and program G16 (Axama Biosystems) with either a control siRNA oligo (−) or a STAT3-specific siRNA oligo (+). After 48 or 72 h, aliquots of cells were harvested for Western Blot analysis of the indicated markers. GAPDH is used as loading control. Triangle symbol in panel (B) indicates cleaved form of PARP-1, the extent of which reflects Caspase 3/7 activity.

FIG. 3A-3B. Overall survival (OS) and event-free survival (EFS) of DLBCL patients according to the IPI risk groups (Low: 0-2; High: 3-5).

FIG. 4A-4B. Overall survival (OS) and event-free survival (EFS) of DLBCL patients according to the cell-of-origin (GCB vs non-GCB/ABC) subtypes.

FIG. 5A-5F. STAT3 activation predicts poor survival in DLBCL patients treated with R-CHOP, especially in patients with non-GCB-DLBCL. The distributions of (A) overall survival (OS) and (B) event-free survival (EFS) of DLBCL patients treated with the R-CHOP regimen are illustrated based on the PY-STAT3 status. In panels (C) and (D), OS and EFS are displayed, respectively, for the non-GCB-DLBCL patients; while OS and EFS of the GCB-DLBCL patients are shown in panels (E) and (F), respectively. The log-rank test was used to evaluate the significance of difference between PY-STAT3 positive and negative phenotypes.

FIG. 6A-6B. STAT3 mRNA expression predicts poor survival in DLBCL patients treated with R-CHOP. The distributions of (A) OS and (B) EFS of patients treated with the R-CHOP regimen are illustrated for these three groups.

FIG. 7A-7C. Generation of a 33-gene PY-STAT3 signature from gene expression profiling of cell lines and clinical samples. (A) The Bayes method with leave-one-out cross-validation was used to train a gene signature for the best prediction of PY-STAT3 expression in patient samples. The STAT3 mRNA expression, PY-STAT3 phenotype, and the DLBCL subtype of each case are shown at the bottom of the panel. (B) The scatter plot shows STAT3 mRNA and PY-STAT3 expression for the 30 cases. (C) Relative mRNA expression of the 33 PY-STAT3 signature genes in the four ABC-DLBCL cell lines. The Module A and Module B genes are indicated. WT and KD indicate knock-down experiment performed with either control (wild type, WT) or STAT3 siRNA (knock down, KD) oligos, respectively. Missing data in Ly3 is due to the absence of certain probes in an earlier version of the Affymetrix microarray (U133A).

FIG. 8A-8B. Validation of the 33-gene PY-STAT3 signature by quantitative reverse transcriptase PCR (qRT-PCR). A total of 10 genes (6 in Module A and 4 in Module B) were selected for this assay. Ly10 (A) and Pfeiffer cells (B) were transfected with either a control siRNA oligo (ctrli) or a STAT3-specific siRNA oligo (STAT3i). Forty-eight hours after transfection, RNA samples were prepared and used for qRT-PCR. mRNA levels in the STAT3 knock-down cells were normalized to that in control cells (set as 1.0). Plotted in the graph are the mean and standard deviation of two duplicate samples.

FIG. 9A-9G. The 33-gene PY-STAT3 signature stratified 233 DLBCL cases treated with R-CHOP into 4 clusters with different immunophenotypes and clinical outcomes. (A) Unsupervised clustering based on the PY-STAT3 signature. Four clusters were obtained by the clustering. Module A and Module B genes were marked by vertical bars on the right of the heat map. The levels of STAT3 mRNA, BCL6 and MUM1 protein, and the DLBCL subtype of each case were labeled. The relative expression of 5 GEP signatures and the Survival Predictive Score for each case are labeled below the heat map as well. (B) OS distributions of the 4 PY-STAT3 clusters. (C) OS distributions of patients in Cluster 4 are compared between the BCL6 negative (n=12) and positive (n=9) groups. (D) Histogram of the median Survival Predictive Score for the 4 PY-STAT3 clusters. Error bar indicates standard error for each group. The P-values are based on two-tailed t-test. (E-G) The heap-map of relative mRNA expression for (E) the Pan-T-Cell signature, (F) the proliferation signature, and (G) the plasmablastic signature of the ABC-DLBCL patients in Cluster 3 (n=62) vs Cluster 4 (n=25). The histogram shows the median of the relative expression of each signature. Error bar indicates standard error for each group.

FIG. 10A-10C. The 33-gene PY-STAT3 signature stratified a 181-case cohort treated with CHOP into four clusters with different immunophenotypes and clinical outcomes. (A) Unsupervised clustering was performed based on the PY-STAT3 gene signature and 4 clusters were obtained. The 4 clusters are displayed according to the expression pattern of Module A and Module B genes on the R-CHOP dataset. Below the heat map, the STAT3 mRNA expression and DLBCL subtypes of each case are labeled. The relative expression of Pan-T-cell signature, plasmablastic signature, proliferative signature, and the survival predictive score for each case are labeled as well. (B) Histogram of median survival predictive score for each cluster is displayed. The P-values demonstrate the significance of difference between Cluster 4 and the other clusters. Error bar indicates standard error for each group. (C) Distributions of overall survival of the four clusters.

FIG. 11A-11D. Cluster-specific comparison of survival outcomes between CHOP and R-CHOP treatment. The 33-gene PY-STAT3 signature was applied to the 181-case CHOP cohort and 233-case R-CHOP cohort as described in FIGS. 9 and 10.

FIG. 12. Development of a PY-STAT3-based 11 gene signature to predict survival outcome of DLBCL patients treated with R-CHOP. Heat-map shows the expression pattern STAT3 candidate genes between PY-STAT3 positive and negative cases. PY-STAT3 IHC scores and STAT3 mRNA levels were illustrated in the top of the heat-map. Green and red colors indicate relatively low and high expression, respectively, in the heat-map.

FIG. 13. Expression of HSD17B4, MT1X, RHEB and SLA were down-regulated following STAT3 knockdown (KD) by siRNA in Ly10 and Pfeiffer cells. Relative mRNA expression was evaluated by qRT-PCR. The measurement was illustrated as the mean expression plus the standard error in two biological replicates. The mean values in the control siRNA treated samples were set to 1.00.

FIG. 14A-14D. A 11-gene PY-STAT3 signature was able to predict the clinical outcome of DLBCL patients in the entire cohort (A, B), as well as those in the ABC-DLBCL subgroup (C, D). OS and EFS distributions are illustrated based on the expression quartiles of the 11-gene predictor.

FIG. 15. Overall survival distribution of DLBCL patients treated with the CHOP regimen is shown according to the average expression of the 11-gene predictor. Low,<mean−one S.D.; High,>mean+one S.D.; intermediate, the remaining of the cases.

FIG. 16A-16D. The PY-STAT3+/BCL6-phenotype predicts the worst survival in patients treated with CHOP. The distributions of (A) OS and (B) EFS of patients treated with the CHOP regimen, and the distributions of (C) OS and (D) EFS of patients treated with the R-CHOP regimen are illustrated based on the PY-STAT3 and BCL6 phenotypes. Legends for the 4 phenotypes are shown at the bottom of the figure.

### DETAILED DESCRIPTION OF THE INVENTION

The present invention provides a method of classifying a human patient with diffuse large B-cell lymphoma (DLBCL), the method comprising determining mRNA expression levels of human genes in a DLBCL biopsy specimen from the patient, wherein the genes comprise HSD17B4, RNF149, ZNF805, SLC2A13, RHEB, MT1X, NAT8L, C15orf29, ZNF420, PCNX and SLA (i.e., the “11 gene signature”), so as to classify the DLBCL patient based on expression levels.

For example, the patient can be classified into a subgroup by comparing the expression of this 11-gene signature from the patient with the expression of the same signature from a cohort of DLBCL patients who have already been classified into expression subgroups. Preferred subgroups include quartiles. A patient classified into the bottom 50% subgroup has a more favorable outcome of survival compared to patients in the top gene expression quartile.

For another example, in the non-GCB/ABC subgroup, a patient classified into the bottom gene expression quartile has a more favorable survival outcome compared to patients in the other quartile subgroups.

The invention also provides a gene expression signature that is predictive of activation of signal transducer and activator of transcription 3 (STAT3), wherein the profile comprises expression of a plurality of, or all of, the following genes: HSD17B4, RNF149, ZNF805, SLC2A13, RHEB, MT1X, NAT8L, C15orf29, ZNF420, PCNX and SLA.

The expression levels of all genes described in the present invention can be normalized to the level of expression of a “housekeeping gene” that is required for the maintenance of basic cellular function. Examples of housekeeping genes include, but are not limited to, ACTB, GAPDH, and STAT1.

An average expression of the gene signature can be obtained, for example, by taking normalized microarray signals for each probe (e.g., as in Table 7), and calculating the mean value.

The invention also provides a method of classifying a human patient with diffuse large B-cell lymphoma (DLBCL), the method comprising

determining mRNA expression levels of human genes in a DLBCL biopsy specimen from the patient, wherein the genes comprise Module A genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1, and Module B genes BTLA, C13orf18, CFLAR, EV12A, HIST2H2AA3, IL16, IL2RA and PTGER4; and

determining the expression levels of Module A genes and Module B genes so as to classify the DLBCL patient based on the expression levels.

Preferably, the genes for which expression is determined are predictive of activation of signal transducer and activator of transcription 3 (STAT3).

The DLBCL patient can be classified into a subgroup by comparing the expression of Module A and Module B genes from the patient with the expression of the same gene signature from a cohort of DLBCL patients who have already been classified into expression subgroups. The DLBCL patient can be classified into one of four clusters depending on the levels of expression of the Module A genes and of the Module B genes. For example, the patient can be classified in Cluster 1 if the majority of genes in Module A is downregulated and if the majority of genes in Module B is downregulated; the patient can be classified in Cluster 2 if the majority of genes in Module A is upregulated and if the majority of genes in Module B is not upregulated; the patient can be classified in Cluster 3 if the majority of genes in Module A is upregulated and if the majority of genes in Module B is upregulated; and/or the patient can be classified in Cluster 4 if the majority of genes in Module A is not upregulated and if the majority of genes in Module B is upregulated.

The invention provides a method of classifying a human patient with diffuse large B-cell lymphoma (DLBCL), the method comprising

determining mRNA expression levels of human genes in a DLBCL biopsy specimen from the patient, wherein the genes comprise Module A genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1, and Module B genes BTLA, C13orf18, CFLAR, EV12A, HIST2H2AA3, IL16, IL2RA and PTGER4; and

comparing the expression levels of Module A genes and Module B genes from the patient with the expression of the Module A genes and Module B genes from a cohort of DLBCL patients who have already been classified into expression subgroups,

wherein the patient is classified in Cluster 1 if the majority of genes in Module A is downregulated and if the majority of genes in Module B is down-regulated;

wherein the patient is classified in Cluster 2 if the majority of genes in Module A is upregulated and if the majority of genes in Module B is not upregulated;

wherein the patient is classified in Cluster 3 if the majority of genes in Module A is upregulated and if the majority of genes in Module B is upregulated; and

wherein the patient is classified in Cluster 4 if the majority of genes in Module A is not upregulated and if the majority of genes in Module B is upregulated,

so as to classify the DLBCL patient based on the expression levels.

The patient can be classified into one of four clusters, for example, by comparing the expression of Module A genes and Module B genes from the patient with the expression of Module A genes and Module B genes from a cohort of DLBCL patients who have already been classified into one of the four clusters. For example, this may be performed by comparing the gene expression pattern obtained from the patient to at least the expression profile associated with one of the four clusters, determining the degree of similarity between the gene expression pattern obtained from the patient and the expression profile associated with at least one of the four clusters, and based on the degree of similarity, classifying the patient into one of the four clusters. For example, a high degree of similarity between the gene expression pattern obtained from the patient and the expression profile for Cluster 1 will lead to classification of the patient into Cluster 1.

An example of one such cohort of subjects with DLBCL is a cohort of 233 DLBCL cases for which both gene expression profile and clinical data are available (National Center for Biotechnology Information (NCBI, Bethesda Md.) accession number GSE10846). See also Lenz et al. (2008).¹⁴ These subjects were treated with the R-CHOP regimen, which is rituximab (R) in combination with cyclophosphamide, hydroxydaunorubicin (doxorubicin), vincristine, and prednisone (CHOP).

The patient can also be classified into one of four clusters by determining y_{pred} for Module A and y_{pred} for Module B, where y_{pred}=b₀+b₁x₁+b₂x₂+ . . . +bₙxₙ, wherein x₁, x₂ . . . xₙ is the expression value of each gene, and where the coefficients b₀, b₁ . . . +bₙ are set forth in Table 5;

wherein the patient is classified in Cluster 1 if y_{pred} for Module A and y_{pred} for Module B are both negative;

wherein the patient is classified in Cluster 2 if y_{pred} for Module A is positive and if y_{pred} for Module B is negative;

wherein the patient is classified in Cluster 3 if y_{pred} for Module A and y_{pred} for Module B are both positive; and

wherein the patient is classified in Cluster 4 if y_{pred} for Module A is negative and if y_{pred} for Module B is positive.

The invention provides a method of classifying a human patient with diffuse large B-cell lymphoma (DLBCL), the method comprising

determining mRNA expression levels of human genes in a DLBCL biopsy specimen from the patient, wherein the genes comprise Module A genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1, and Module B genes BTLA, C 13orf18, CFLAR, EV12A, HIST2H2AA3, IL16, IL2RA and PTGER4; and

classifying the patient into one of four clusters by determining ypred for Module A and ypred for Module B, where ypred=b0+b1x1+b2×2+ . . . +bnxn, where x1, x2 . . . xn is the expression value of each gene, and where the coefficients b0, b1 . . . +bn are set forth in Table 5;

wherein the patient is classified in Cluster 1 if ypred for Module A and ypred for Module B are both negative;

wherein the patient is classified in Cluster 2 if ypred for Module A is positive and if ypred for Module B is negative;

wherein the patient is classified in Cluster 3 if ypred for Module A and ypred for Module B are both positive; and

wherein the patient is classified in Cluster 4 if ypred for Module A is negative and if ypred for Module B is positive,

so as to classify the DLBCL patient.

The invention provides a method of classifying a human patient with diffuse large B-cell lymphoma (DLBCL), the method comprising determining STAT3 mRNA expression level in a DLBCL biopsy specimen from the patient, and comparing the level of STAT3 mRNA expression from the patient with the level of expression of STAT3 mRNA from a cohort of DLBCL patients, wherein a patient with a level of STAT3 mRNA expression that is greater than 1 standard deviation above the mean level of STAT3 mRNA expression in the cohort has a less favorable survival outcome compared to patients having a level of STAT3 mRNA expression that is less than 1 standard deviation below the mean level of STAT3 mRNA expression in the cohort.

The Entrez Gene (National Center for Biotechnology Information) and HUGO Gene Nomenclature Committee (HGNC) gene identification numbers, and probe set, for Module A and Module B genes are as follows:

| Probe set | Symbol | Entrez_GeneID | Entrez_GeneName | HGNC |
|---|---|---|---|---|
| 91816_f_at | MEX3D | 399664 | MEX3D | 16734; MEX3D |
| 205965_at | BATF | 10538 | BATF | 958; BATF |
| 208683_at | CAPN2 | 824 | CAPN2 | 1479; CAPN2 |
| 200952_s_at | CCND2 | 894 | CCND2 | 1583; CCND2 |
| 205831_at | CD2 | 914 | CD2 | 1639; CD2 |
| 224733_at | CMTM3 | 123920 | CMTM3 | 19174; CMTM3 |
| 201999_s_at | DYNLT1 | 6993 | DYNLT1 | 11697; DYNLT1 |
| 214446_at | ELL2 | 22936 | ELL2 | 17064; ELL2 |
| 201724_s_at | GALNT1 | 2589 | GALNT1 | 4123; GALNT1 |
| 203765_at | GCA | 25801 | GCA | 15990; GCA |
| 204220_at | GMFG | 9535 | GMFG | 4374; GMFG |
| 211275_s_at | GYG1 | 2992 | GYG1 | 4699; GYG1 |
| 210164_at | GZMB | 3002 | GZMB | 4709; GZMB |
| 221760_at | MAN1A1 | 4121 | MAN1A1 | 6821; MAN1A1 |
| 204326_x_at | MT1X | 4501 | MT1X | 7405; MT1X |
| 217744_s_at | PERP | 64065 | PERP | 17637; PERP |
| 207002_s_at | PLAGL1 | 5325 | PLAGL1 | 9046; PLAGL1 |
| 214617_at | PRF1 | 5551 | PRF1 | 9360; PRF1 |
| 209515_s_at | RAB27A | 5873 | RAB27A | 9766; RAB27A |
| 217728_at | S100A6 | 6277 | S100A6 | 10496; S100A6 |
| 213572_s_at | SERPINB1 | 1992 | SERPINB1 | 3311; SERPINB1 |
| 238480_at | TTC39C | 125488 | TTC39C | 26595; TTC39C |
| 206698_at | XK | 7504 | XK | 12811; XK |
| 219836_at | ZBED2 | 79413 | ZBED2 | 20710; ZBED2 |
| 225959_s_at | ZNRF1 | 84937 | ZNRF1 | 18452; ZNRF1 |
| 236226_at | BTLA | 151888 | BTLA | 21087; BTLA |
| 219471_at | C13orf18 | 80183 | C13orf18 | 20420; |
| | | | | C13orf18 |
| 210563_x_at | CFLAR | 8837 | CFLAR | 1876; CFLAR |
| 204774_at | EVI2A | 2123 | EVI2A | 3499; EVI2A |
| 218280_x_at | HIST2H2AA3 | 8337 | HIST2H2AA3 | 4736; |
| | | | | HIST2H2AA3 |
| 209827_s_at | IL16 | 3603 | IL16 | 5980; IL16 |
| 206341_at | IL2RA | 3559 | IL2RA | 6008; IL2RA |
| 204897_at | PTGER4 | 5734 | PTGER4 | 9596; PTGER4 |

The levels of expression of these genes can be determined, for example, using standard gene expression microarray procedures. A microarray contains, for example, a plurality of nucleic acid probes coupled to the surface of a substrate in different known locations. Microarrays are well known in the art and can be obtained, for example from Affymetrix (Santa Clara, Calif.). Gene expression data can also be obtained using, for example, reverse transcription-polymerase chain reaction (RT-PCR).

Classification of the DLBCL patient can aid in predicting the treatment that may be most beneficial for the patient.

In one embodiment, a patient classified in Cluster four is predicted to be the least likely to benefit from therapy with rituximab in combination with cyclophosphamide, doxorubicin, vincristine, and prednisone (R-CHOP), compared to a patient in Cluster one, two or three.

In one embodiment, a DLBCL patient undergoing therapy with a combination of cyclophosphamide, doxorubicin, vincristine, and prednisone (CHOP) classified in Cluster two is predicted to have a more favorable likelihood of survival compared to a patient classified in Cluster three.

In one embodiment, a patient classified in Cluster one or three is predicted to benefit more from therapy with rituximab in combination with cyclophosphamide, doxorubicin, vincristine, and prednisone (R-CHOP), compared to a patient in Cluster two or four.

The invention also provides a method of determining the prognosis of a diffuse large B-cell lymphoma (DLBCL) patient undergoing treatment with rituximab in combination with cyclophosphamide, doxorubicin, vincristine, and prednisone (R-CHOP), or treatment with rituximab in combination with cyclophosphamide, mitoxantrone, vincristine, and prednisone (R-CNOP), the method comprising determining the level of phospho-Tyr705-STAT3 (PY-STAT3) in a DLBCL biopsy specimen from the patient using immunohistochemistry, wherein PY-STAT3 positivity predicts a poor likelihood of survival in comparison to a patient with PY-STAT3 negativity.

The invention also provides a method of determining the prognosis of a diffuse large B-cell lymphoma (DLBCL) patient undergoing treatment with rituximab in combination with cyclophosphamide, doxorubicin, vincristine, and prednisone (R-CHOP), or treatment with rituximab in combination with cyclophosphamide, mitoxantrone, vincristine, and prednisone (R-CNOP), the method comprising determining the level of phospho-Tyr705-STAT3 (PY-STAT3) in a DLBCL biopsy specimen from the patient using immunohistochemistry; and

determining PY-STAT3 positivity or negativity by scoring the intensity of PY-STAT3 staining using a 4-tiered scale (0, 3, 6, 9), scoring the percentage of PY-STAT3 stained DLBCL tumor cells using a 10-tiered scale (0-9), and multiplying the two scores together to obtain a case score for the patient, where a case score with a value of 15 or greater is considered positive and a case score with a value below 15 is considered negative;

wherein PY-STAT3 positivity predicts a poor likelihood of survival in comparison to a patient with PY-STAT3 negativity.

An antibody for PY-STAT3 can be obtained, for example, from Cell Signaling Technology (Catalog #9131). Double immunostaining for PY-STAT3 and CD20 can be performed to obtain tumor cell-specific PY-STAT3 expression. CD20 antibody can be obtained, for example, from Dako, Carpinteria, Calif. or from LabVision (Clone L26).

The patient can be a non-germinal center B-cell-like (non-GCB) DLBCL patient. DLBCL patients can be classified as germinal center B-cell-like- (GCB-) and non-GCB-DLBCL patients using, for example, expression of CD10, BCL6 and MUM1 as described by Hans et al. (2004).²²

The invention further provides a method of determining the prognosis of a diffuse large B-cell lymphoma (DLBCL) patient undergoing treatment with a combination of cyclophosphamide, doxorubicin, vincristine, and prednisone (CHOP), or with a combination of cyclophosphamide, mitoxantrone, vincristine, and prednisone (CNOP), the method comprising determining the level of phospho-Tyr705-STAT3 (PY-STAT3) and the level of BCL6 in a DLBCL biopsy specimen from the patient using immunohistochemistry, wherein PY-STAT3 positivity and BCL6 negativity predicts a poor likelihood of survival in comparison to a patient who is not PY-STAT3 positive and BCL6 negative.

Preferably, PY-STAT3 positivity or negativity is determined by scoring the intensity of PY-STAT3 staining using a 4-tiered scale (0, 3, 6, 9), scoring the percentage of PY-STAT3 stained DLBCL tumor cells using a 10-tiered scale (0-9), and multiplying the two scores together to obtain a case score for the patient, where a case score with a value of 15 or greater is considered positive and a case score with a value below 15 is considered negative. Preferably, the patient is considered BCL6 positive if 30% or more of the DLBCL tumor cells stain positive for BCL6, and BCL6 negative if less than 30% of the DLBCL tumor cells stain positive for BCL6.

BCL6 antibody can be obtained, for example, from Santa Cruz Biotechnology, Santa Cruz, Calif. (Catalog number sc-858).

The invention provides a method of determining the prognosis of a diffuse large B-cell lymphoma (DLBCL) patient undergoing treatment with a combination of cyclophosphamide, doxorubicin, vincristine, and prednisone (CHOP), or with a combination of cyclophosphamide, mitoxantrone, vincristine, and prednisone (CNOP), the method comprising determining the level of phospho-Tyr705-STAT3 (PY-STAT3) and the level of BCL6 in a DLBCL biopsy specimen from the patient using immunohistochemistry; and

determining PY-STAT3 positivity or negativity by scoring the intensity of PY-STAT3 staining using a 4-tiered scale (0, 3, 6, 9), scoring the percentage of PY-STAT3 stained DLBCL tumor cells using a 10-tiered scale (0-9), and multiplying the two scores together to obtain a case score for the patient, where a case score with a value of 15 or greater is considered positive and a case score with a value below 15 is considered negative, and wherein the patient is considered BCL6 positive if 30% or more of the DLBCL tumor cells stain positive for BCL6, and BCL6 negative if less than 30% of the DLBCL tumor cells stain positive for BCL6;

wherein PY-STAT3 positivity and BCL6 negativity predicts a poor likelihood of survival in comparison to a patient who is not PY-STAT3 positive and BCL6 negative.

For the methods disclosed herein, the steps of determining mRNA expression levels, determining the level of phospho-Tyr705-STAT3 (PY-STAT3), and determining the level of BCL6 in a DLBCL biopsy specimen from a patient require an experimental determination that involves the use of a machine and/or involves a physical and/or chemical transformation.

The invention also provides a gene expression profile or signature that is predictive of activation of signal transducer and activator of transcription 3 (STAT3), wherein the profile comprises expression of a plurality of, or all of, the following genes: MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

STAT3 activation can be positively correlated with expression of one or more of, or with all of, MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1.

STAT3 activation can be positively correlated with expression of one or more of, or with all of, MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The gene expression profile can be obtained by determining mRNA expression in a human diffuse large B-cell lymphoma (DLBCL) biopsy specimen. The biopsy specimen can be, for example, from a subject diagnosed as having DLBCL before the subject undergoes treatment for DLBCL, e.g., prior to undergoing treatment with CHOP or R-CHOP.

The invention provides a gene expression profile or signature for classifying a human patient with diffuse large B-cell lymphoma (DLBCL), where the signature comprises nucleic acid probes for genes HSD17B4, RNF149, ZNF805, SLC2A13, RHEB, MT1X, NAT8L, C15orf29, ZNF420, PCNX and SLA.

The invention also provides a gene expression profile or signature for classifying a human patient with diffuse large B-cell lymphoma (DLBCL), where the signature comprises nucleic acid probes for genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The invention provides a microarray for classifying a human patient with diffuse large B-cell lymphoma (DLBCL), where the microarray comprises nucleic acid probes for genes HSD17B4, RNF149, ZNF805, SLC2A13, RHEB, MT1X, NAT8L, C15orf29, ZNF420, PCNX and SLA.

The invention also provides a microarray for classifying a human patient with diffuse large B-cell lymphoma (DLBCL), where the microarray comprises nucleic acid probes for genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

The microarray can comprise probes attached, for example, via surface engineering to a solid surface by a covalent bond to a chemical matrix (via, in non-limiting examples, epoxy-silane, amino-silane, lysine, polyacrylamide). Suitable solid surface can be, in non-limiting examples, glass or a silicon chip, a solid bead forms of, for example, polystyrene. Microarrays can include solid-phase microarrays and bead microarrays. In an embodiment, the microarray is a solid-phase microarray. In an embodiment, the microarray is a plurality of beads microarray. In an embodiment, the microarray is a spotted microarray. In an embodiment, the microarray is an oligonucleotide microarray. The oligonucleotide probes of the microarray may be of any convenient length necessary for unique discrimination of targets. In non-limiting examples, the oligonucleotide probes are 20 to 30 nucleotides in length, 31 to 40 nucleotides in length, 41 to 50 nucleotides in length, 51 to 60 nucleotides in length, 61 to 70 nucleotides in length, or 71 to 80 nucleotides in length. In an embodiment, the target sample, or nucleic acids derived from the target sample, such as mRNA or cDNA, are contacted with a detectable marker, such as one or more fluorophores, under conditions permitting the fluorophore to attach to the target sample or nucleic acids derived from the target sample. In non-limiting examples the fluorophores are cyanine 3 or cyanine 5. In an embodiment, the target hybridized to the probe can be detected, for example, by conductance, MS, or electrophoresis. The microarray can be manufactured by any method known in the art including, for example, by photolithography, pipette, drop-touch, piezoelectric (ink-jet), and electric techniques.

STAT3 activation for prognosis of patients with DLBCL can be combined with the use of additional biomarkers, e.g., BCL6 expression for the CHOP treatment and the non-GCB immunophenotype for the R-CHOP regimen.

This invention will be better understood from the Examples, which follow. However, one skilled in the art will readily appreciate that the specific methods and results discussed are merely illustrative of the invention as described more fully in the claims that follow thereafter.

### EXPERIMENTAL DETAILS

### Example A

### PY-STAT3-Based Method and the 33 Gene Model

### INTRODUCTION

A retrospective analysis of DLBCL patients treated with R-CHOP was performed focusing on understanding the prognostic significance of STAT3 activation. By quantitating the levels of phospho-Tyr705-STAT3 (PY-STAT3) in tumor cells, it was demonstrated that PY-STAT3 positivity predicted poor survival in DLBCL patients, especially in the non-GCB subgroup. In addition, a 33-gene PY-STAT3 gene expression profiling (GEP) signature can stratify R-CHOP treated DLBCL patients into four subgroups with different immunophenotypes and survival outcomes.

### Methods

Patient Information and Gene Expression Profiles:

The study population included 309 patients with de novo DLBCL who were diagnosed and treated with rituximab plus standard CHOP or CHOP-like therapy (R-CHOP). Among these patients, 99 were treated at the Nebraska Lymphoma Study Group, while the rest of the cases were treated at the other LLMPP affiliated institutions. This study was approved by the institutional review boards of University of Nebraska Medical Center and of other respective institutions, and all patients gave written informed consent. Gene expression profiling (GEP) information for 222 of these patients (contain 12 Nebraska cases) was previously published and publicly available.¹⁴ The mRNA expression of STAT3 was evaluated using the averaged intensity of three probe-sets (208991_at, 208992_s_at, and 225289at) from the GEP datasets. Expression of the 3 probe-sets significantly correlated with each other (Pearson correlation, P<0.001).

Tissue Microarray and Immunohistochemistry (IHC):

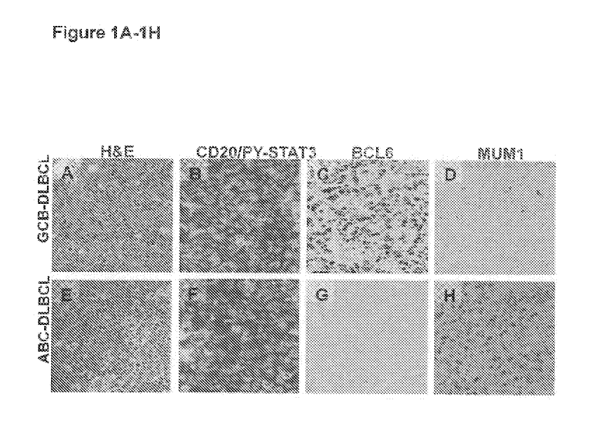

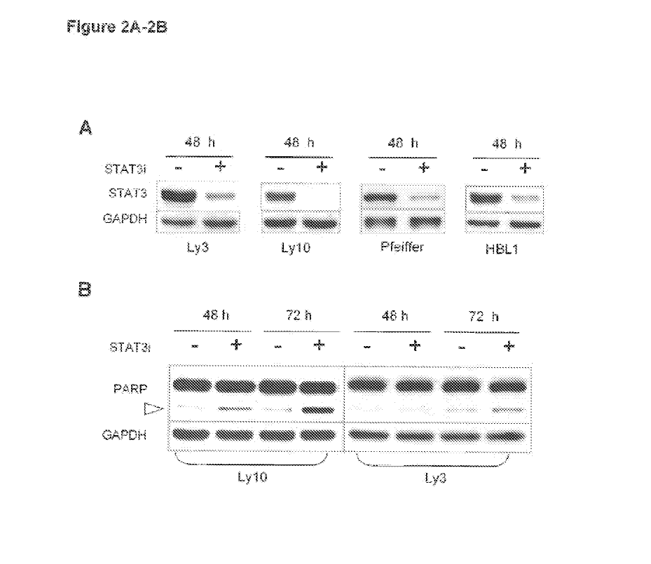

The methods of tissue processing and tissue microarray (TMA) construction have been described previously.¹³ A classification of GCB- and non-GCB-DLBCL was utilized based on an algorithm described by Hans et al.²² Double immunostaining for PY-STAT3 and CD20 was performed to measure tumor cell-specific PY-STAT3 expression (FIG. 1). The percentage and intensity of PY-STAT3 staining were independently scored. A 4-tiered scale (0, 3, 6, 9) was used to score the staining intensity and a 10-tiered scale (0-9) was used to grade the percentage of PY-STAT3 positive tumor cells. The product of both was used as a case score and a value of 15 or greater was considered positive (e.g. equal or greater than 50% positive tumor B cells with intensity of 3 or 30% positive cells with intensity of 6)⁸. Internal positive controls for each TMA core were required for interpretation. The samples were analyzed independently by three hematopathologists, and disagreements were resolved by joint review on a multi-headed microscope.

STAT3 siRNA Experiment:

SiRNA-mediated knock-down experiments were performed using 4 human PY-STAT3 positive DLBCL cell lines: Ly3, Ly10, HBL1, and Pfeiffer. The first three lines express constitutively activated STAT3^{8,9} while Pfeiffer has moderate levels STAT3 activation (data not shown). All cell lines were transiently transfected with either STAT3 siRNA or a control oligo-nucleotides in triplicate as described previously.⁸ Substantial knock-down of the STAT3 protein was achieved at 48 hrs. At this time, endogenous STAT3 was significantly down-regulated with little or no signs of apoptosis (FIG. 2). Total RNA was prepared and used for GEP analysis using Affymetrix (Santa Clara, Calif.) HG U133A (Ly3) or HG U133 Plus2 (HBL1, Ly10, and Pfeiffer) arrays following the standard protocol.

Generation of the 33 Gene PY-STAT3 Signature:

GEP data from the STAT3 siRNA experiment were extracted and normalized using the BRB-Array Tools (National Cancer Institute, NIH). The SAM²³ algorithm was used to identify genes that were differentially expressed between STAT3 and control siRNA treated samples. To develop a STAT3-based gene expression signature that has prognostic value, genes that were significantly altered by STAT3 siRNA and differentially expressed between PY-STAT3 positive and negative cases (P<0.05) were used. Semi-supervised prediction (SSP) method was used to regress the differentially expressed STAT3 targets by patient overall survival based on the Cox proportional hazard model with a significance of 0.05.²⁴ The leave-one-out approach was used for cross-validation.²⁵

Survival Analysis:

Clinical and pathological characteristics of patients in different categories were compared by chi-square test. Kaplan-Meier method was used to estimate the overall survival (OS) and event-free survival (EFS) distributions, and the differences were compared using the log-rank test. Cox proportional-hazards regression model was used to evaluate predictors of the survival distributions while adjusting for international prognostic index (IPI) and COO subgroups. All reported P-values are two-sided and those <0.05 were considered statistically significant.

### Results

Clinical Characteristics of Patients:

There were a total of 309 DLBCL cases in this study. The median age of the entire cohort was 62.2 years (range, 16.7 to 92.0 years), and the male to female ratio was 1.4 (180/129). Of the 185 cases examined for PY-STAT3 by IHC, 69 (37.3%) cases were positive and 116 (62.7%) cases were negative. The clinical features were not significantly different between the PY-STAT3-positive and -negative cases (Table 1), except a weak association of PY-STAT3 with the IPI high-risk (3-5) group (32.8% vs 20.0%, P=0.094).

STAT3 Activation is Significantly Associated with ABC-DLBCL:

The cohort of 87 Nebraska cases only with IHC defined COO subgroups, PY-STAT3 positivity was significantly associated with the non-GCB subgroup compared to the GCB subgroup (63.9%, 23/36 vs 41.1%, 21/51, P=0.037). For the rest cases with GEP defined COO subgroup status, PY-STAT3 was marginally enriched in ABC-DLBCLs relative to the GCB-DLBCL subgroup (33.3%, 14/42 vs 17.8%, 8/45, P=0.096). Among all patients within the Nebraska and LLMPP cohorts, the ABC-DLBCL (or non-GCB) subgroup contained significantly more PY-STAT3 positive cases compared to the GCB-subgroup (47.4%, 37/78 vs 30.2%, 29/96, P=0.030). Since Mum1/IRF4 is a hallmark of ABC-DLBCL, as expected, PY-STAT3 positivity also showed significant association with MUM1/IRF4 (P=0.041, Table 1). Consistent with previous reports on two different cohorts^{8,9}, high level STAT3 mRNA expression preferentially occurred in the ABC subgroup (Table 2).

STAT3 Activation Predicts Poor Survival in DLBCL and ABC-DLBCL:

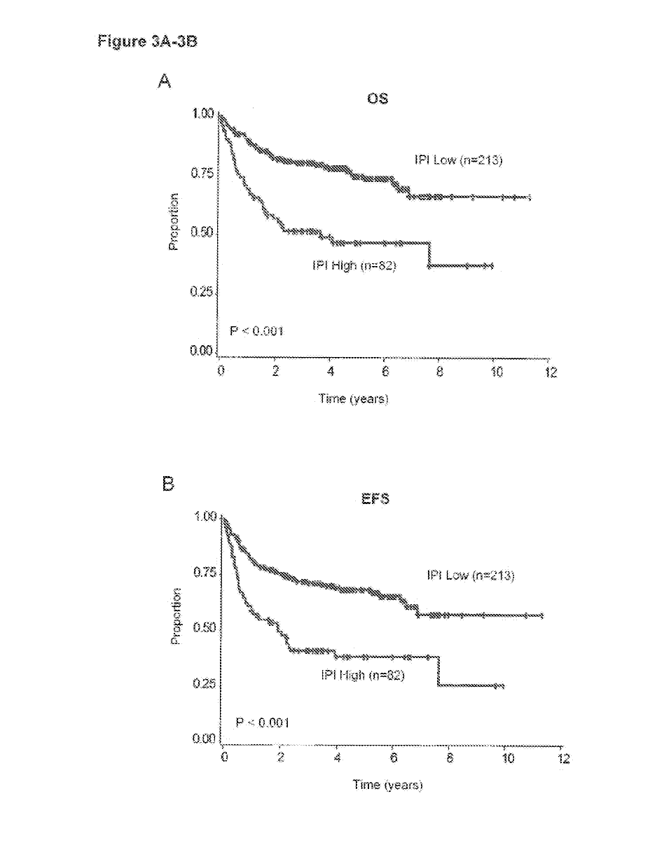

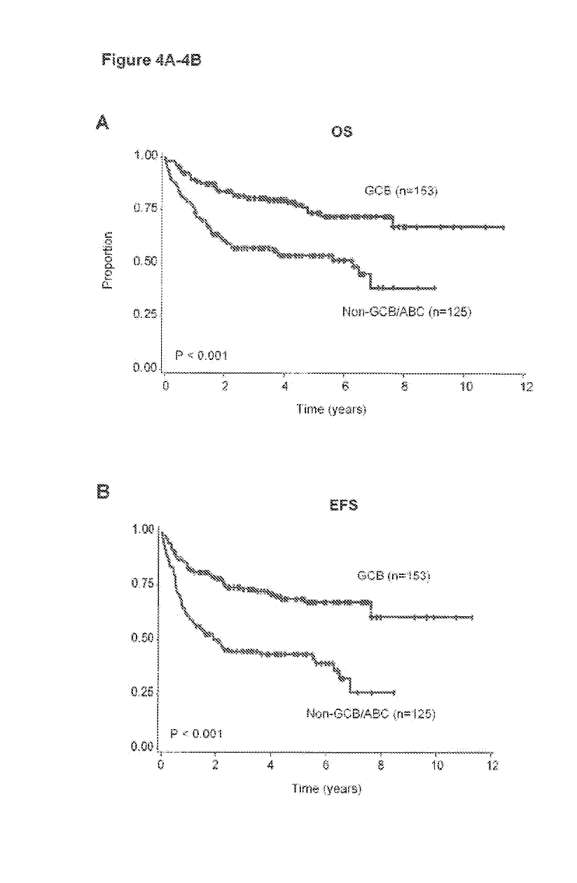

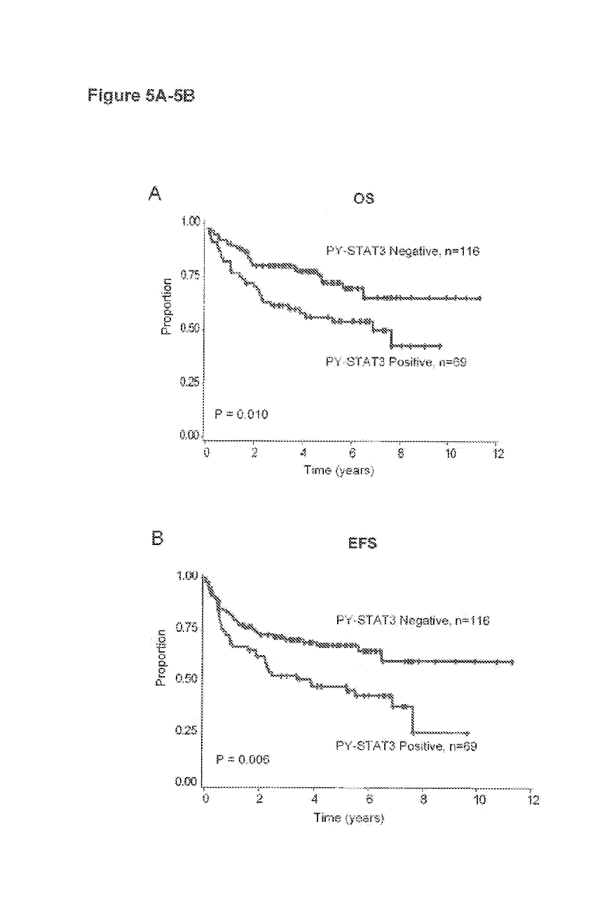

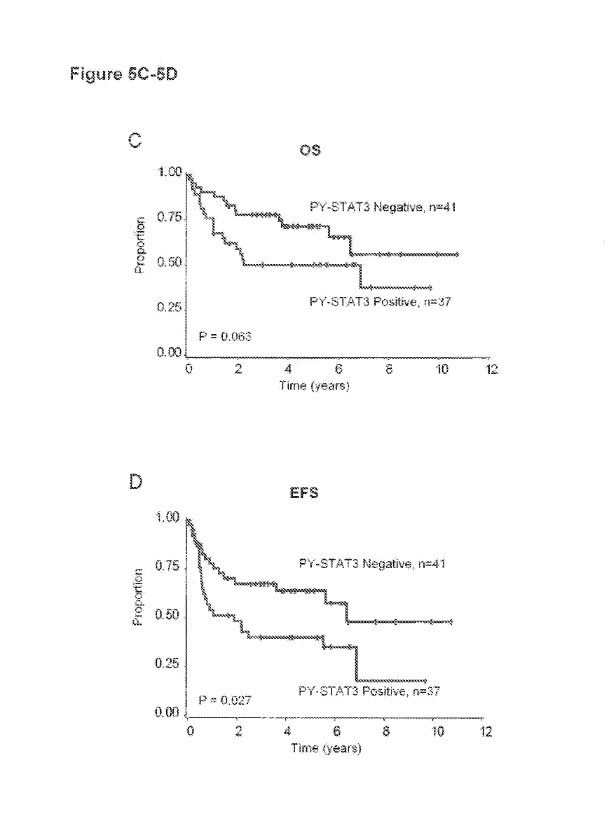

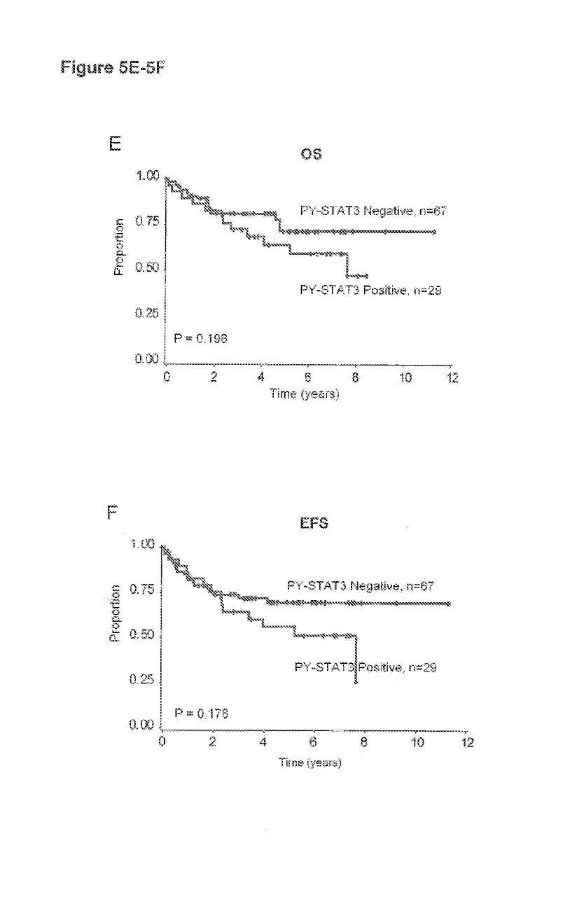

As expected, the IPI and the GCB/non-GCB classifiers (defined by either TMA or GEP) showed significant association with OS and EFS in the entire cohort (FIGS. 3 and 4). When the entire cohort was considered, PY-STAT3 positive cases showed inferior survival compared to the negative cases (P_{OS}=0.010; P_{EFS}=0.006, FIG. 5A-B). PY-STAT3 expression also predicted poor survival in the non-GCB/ABC subgroup (P_{OS}=0.063; P_{EFS}=0.027, FIG. 5C-D), but not in the GCB subgroup (P_{OS}=0.198; P_{EFS}=0.178, FIG. 5E-F). Similar observation was made whether the analysis was performed separately with GEP-defined and IHC defined subgroups. Multivariate analysis was performed using the Cox proportional hazard model. It showed that PY-STAT3 has prognostic significance independent of IPI and COO status (P_{OS}=0.042; P_{EFS}=0.022, Table 3). These results suggest that STAT3 activation in the non-GCB/ABC subgroup identifies a subset of patients who were at high risk when treated with R-CHOP.

Patients with PY-STAT3+/BCL6—Phenotype Had Inferior Survival with the CHOP Regimen:

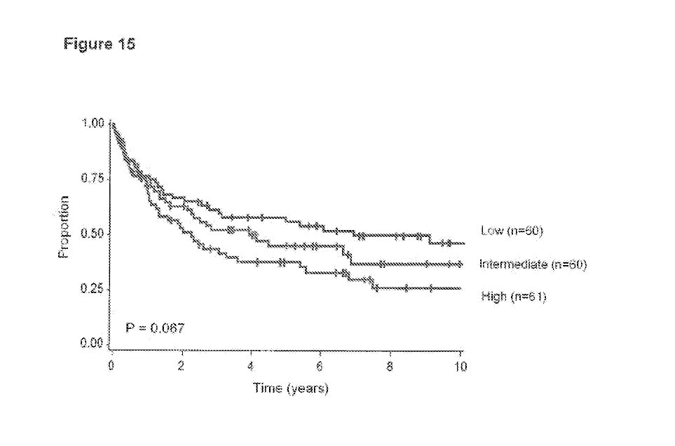

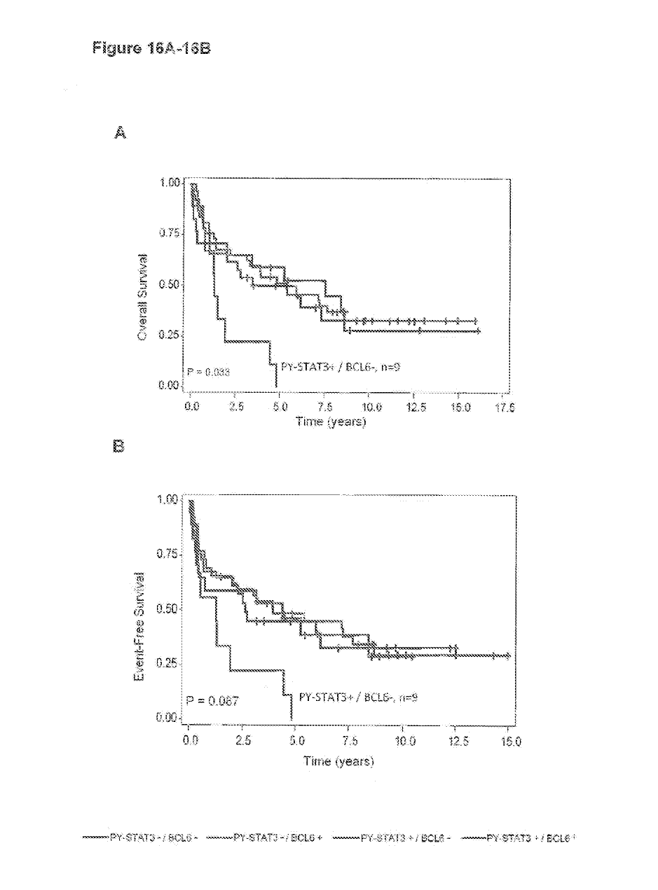

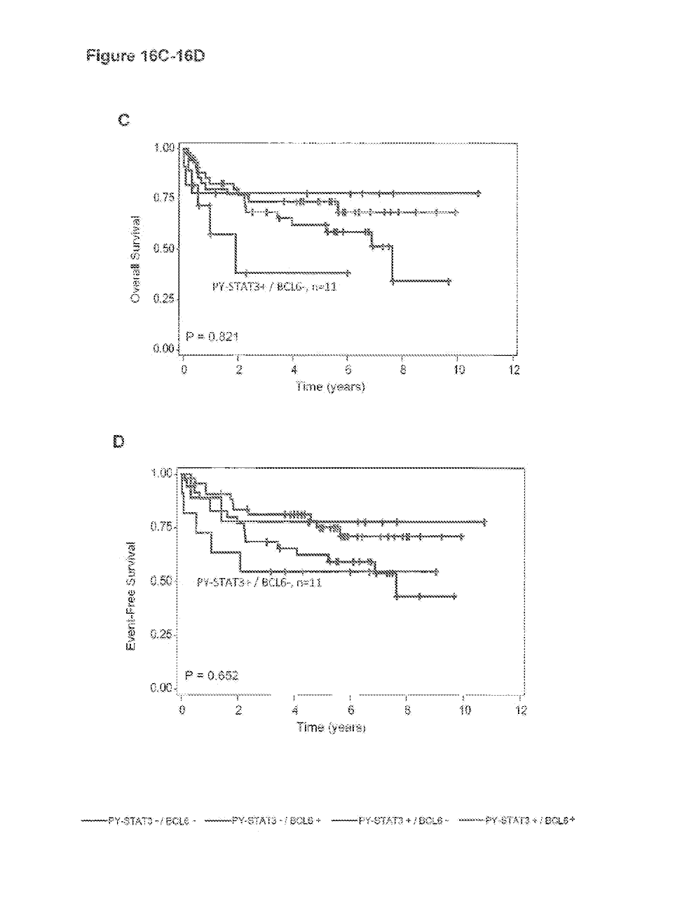

Since BCL6 is an important criterion in the Hans classifier scheme and BCL6 and PY-STAT3 appear to be independently regulated, these two markers were combined in survival analysis (only the Nebraska CHOP and R-CHOP cases were used for this test). In the CHOP group (n=89), 9 patients had PY-STAT3+/BCL6-phenotype and showed a poor OS and EFS (POS=0.033; PEFS=0.087, FIGS. 16A and 16B) compared to the rest of the cohort. All 9 patients were of the non-GCB subtype and died within five years. In the R-CHOP group (n=99), 11 patients had the PY-STAT3+/BCL6-phenotype among which 9 were non-GCB and two were GCB cases. Among the 9 non-GCB patients, 5 died within two years and 4 patients were still alive at the last contact (POS=0.821; PEFS=0.652; FIGS. 16C and 16D). Despite the small number of patients in the two cohorts and relatively short follow-up in the R-CHOP cohort, this result suggests that patients with PY-STAT3+/BCL6-phenotype are at particularly high risk when treated with CHOP but their survival may be significantly improved by the R-CHOP treatment.

High Level STAT3 mRNA is an Adverse Risk Factor in DLBCL:

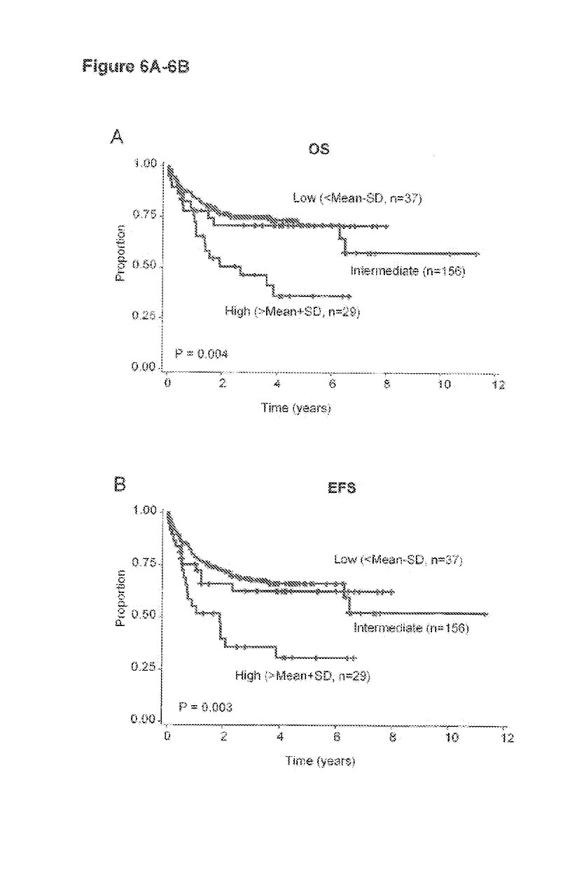

Since the level of STAT3 mRNA significantly correlated with the PY-STAT3 IHC score (Pearson correlation, P<0.001), the prognostic value of this biomarker was also examined DLBCL cases were divided into low (<group mean−standard deviation, S.D, n=37), high (>mean+S.D., n=29), and intermediated (the rest cases, n=156) groups based on the average intensity of the three STAT3 probe-sets (Table 2). Clinical characteristics of patients in these 3 groups were not significantly different. Pathologically, high levels of STAT3 mRNA were correlated with the ABC subtype, Mum1/IRF4 expression, and PY-STAT3 positivity. Similar to the observations on PY-STAT3, cases with high levels of STAT3 mRNA had significantly worse OS and EFS (P_{OS}=0.004; P_{EFS}=0.003, FIG. 6). However, STAT3 mRNA did not show prognostic significance when the cohort was divided into GCB- or ABC-subgroups, likely due to the very small number of the STAT3 high cases in each subgroup (not shown).

A GEP-Based PY-STAT3 Signature is a Predictor of Survival in DLBCL:

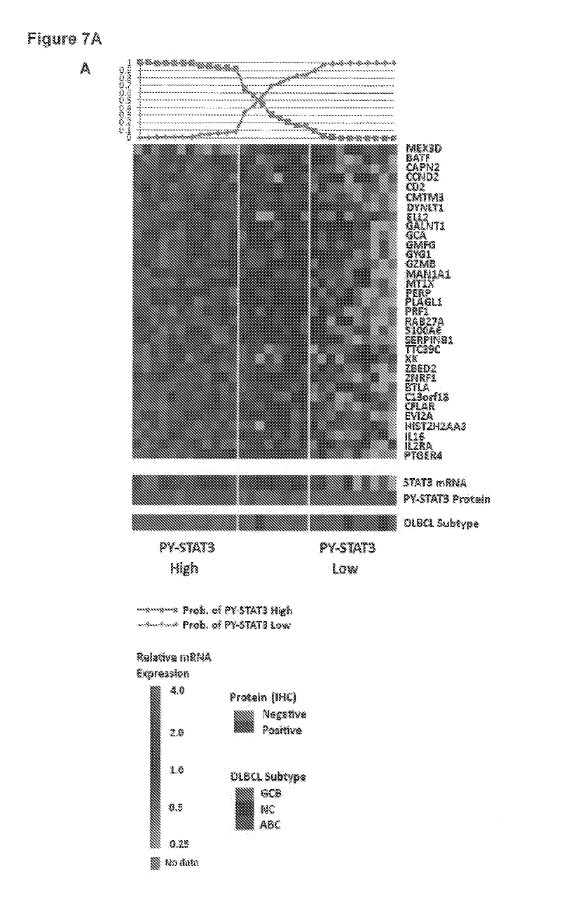

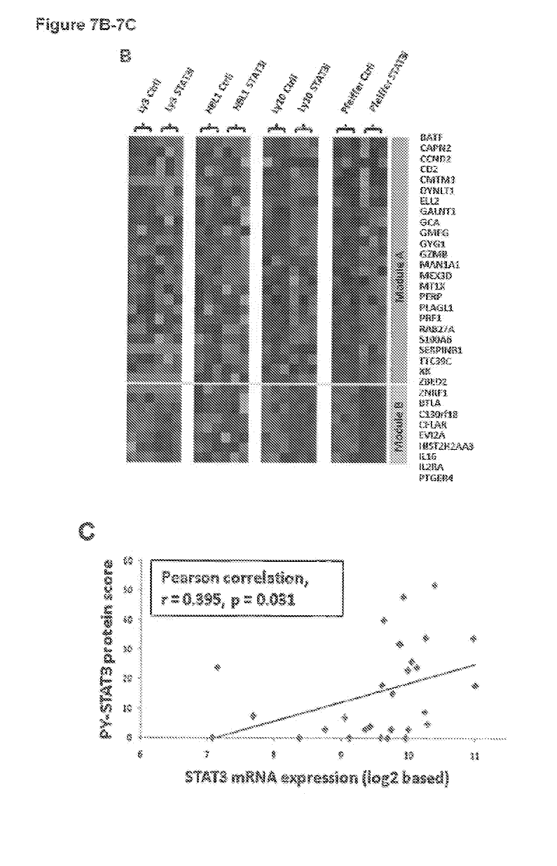

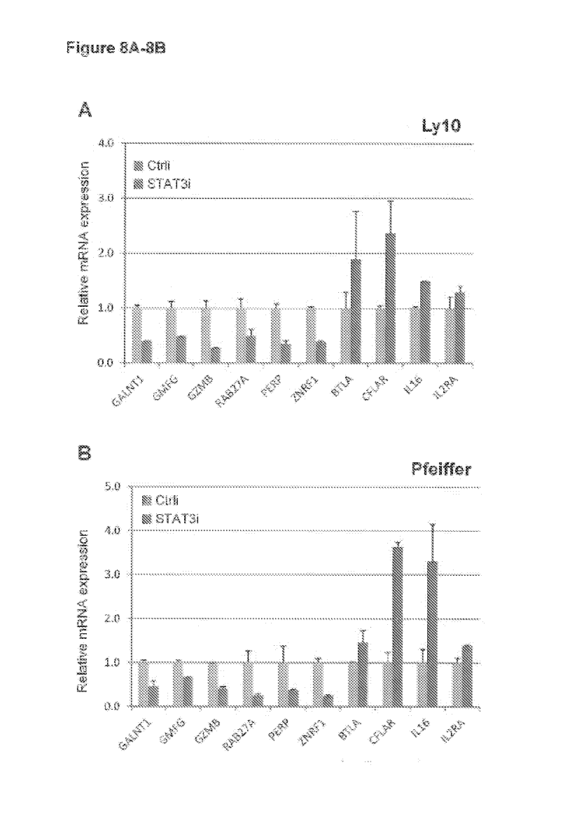

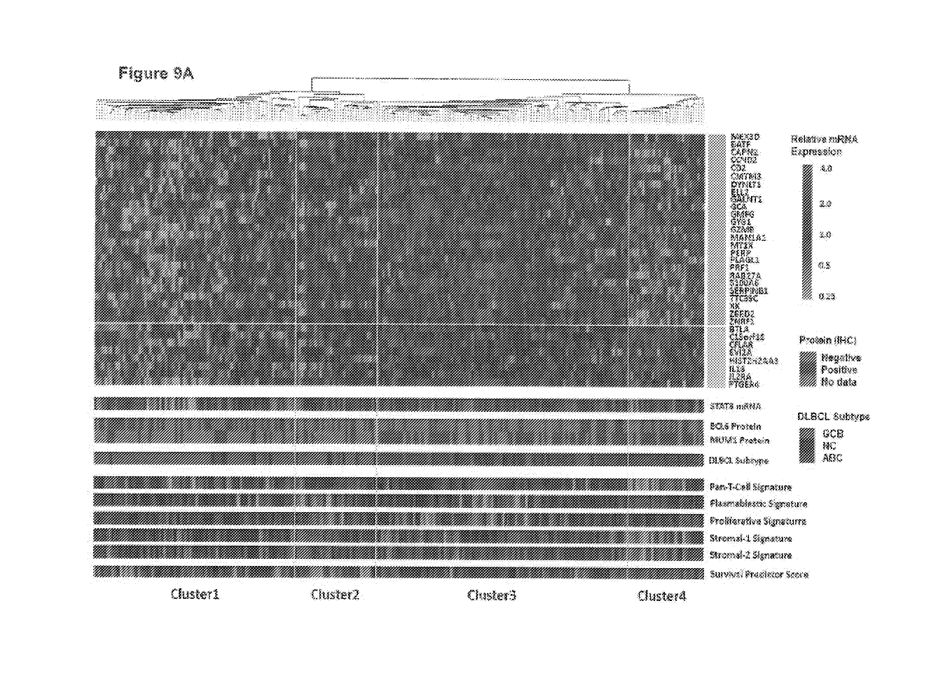

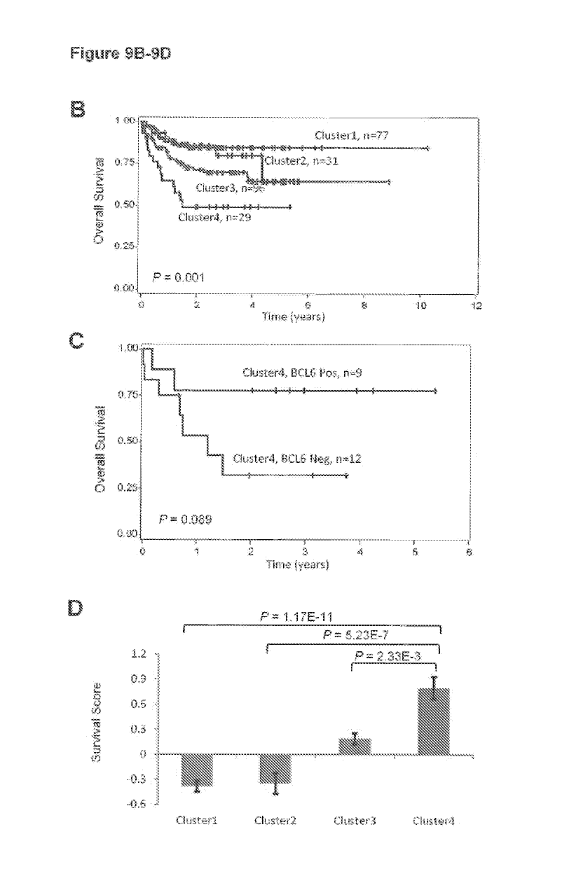

In order to evaluate the generality and reproducibility of the prognostic finding on PY-STAT3, a GEP-based PY-STAT3 signature was constructed based on the test cases described above. This signature was subsequently applied to a large public available GEP dataset that comes with treatment response information. GEP of DLBCL lines was obtained 48 hr after STAT3 siRNA treatment. At this time, endogenous STAT3 was significantly down-regulated with little or no signs of apoptosis based on PARP cleavage (FIG. 2). SAM algorithm was used to identify 1732 genes that were differentially expressed between STAT3 siRNA treated and control cells. Next, to select a subset of genes whose expression best predict STAT3 activation rather than STAT3 expression status, an analysis was conducted of 30 DLBCL cases for which both GEP and IHC-defined PY-STAT3 data were available. By applying the Bayes algorithm, 33 unique genes were identified among the 1732 differentially expressed genes with a predictive power of 95% in cross-validation. Within this PY-STAT3 signature, all 33 genes were positively correlated with PY-STAT3 in the 30 training cases (FIG. 7A). A weak but positive linear correlation was observed between PY-STAT3 and STAT3 mRNA expression (Pearson's correlation, r=0.395, P=0.031, FIG. 7B). However, in the 4 ABC-DLBCL cell lines, the expression of only 25 genes (Module A) was positively correlated with the presence of STAT3 while the other 8 genes (Module B) showed an inverse correlation (FIG. 7C). This result was confirmed by real-time RT-PCR of 10 representative genes, 6 in Module A and 4 in Module B (FIG. 8).

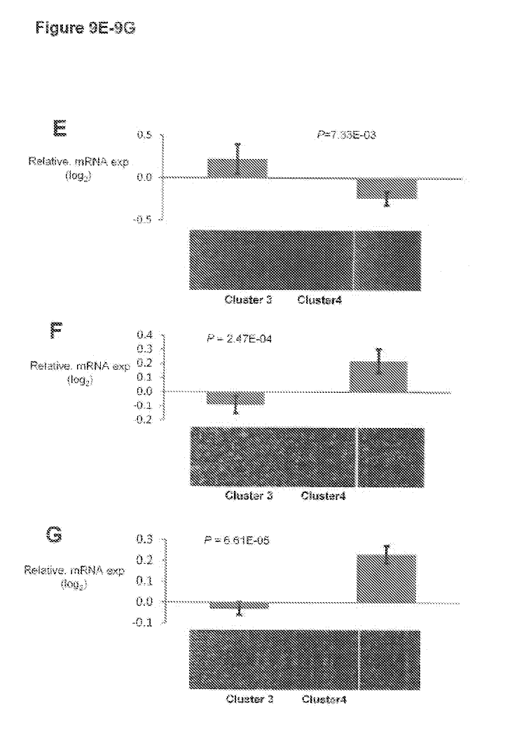

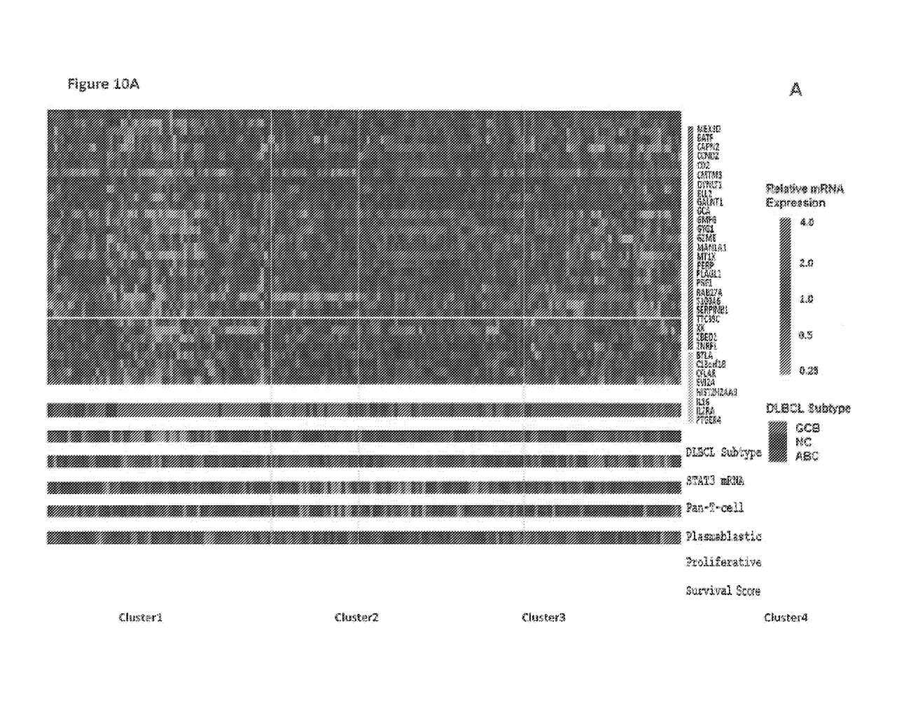

Using an unsupervised hierarchy clustering method, this 33-gene PY-STAT3 signature was applied to the GEP dataset that comprises 233 clinically well-characterized DLBCL cases treated with R-CHOP.¹⁴ The PY-STAT3 signature stratified the cohort into 4 clusters, each corresponding to one of four possible combinations of Module A and Module B (FIG. 9A). Specifically, both Modules were prominently expressed in Cluster 3 and suppressed in Cluster 1. Cluster 2 cases were moderately positive for Module A genes only while the opposite was found in Cluster 4. Since the great majority of Cluster 4 cases were STAT3 mRNA low or negative (FIG. 9A, middle panel), continued expression of Module B genes in Cluster 4 is most likely STAT3-independent. Therefore, both Cluster 1 and Cluster 4 are interpreted to contain either no or low STAT3 activity while Cluster 2 and 3 harbor low and high levels of STAT3 activation, respectively. Interestingly, nearly all cases within Cluster 1/2 belonged to the GCB subgroup, while the large majority of Cluster 3/4 cases were ABC-DLBCL. Of note, the GEP-based observation that the majority (˜65%) of ABC-DLBCL cases bear prominent PY-STAT3 signature while a minor fraction of GCB-DLBCLs (˜30%) also display signs of STAT3 activation is entirely consistent with the IHC-based findings (Table 1). Most importantly, the four clusters had significantly different OS, and cluster 4 had the least favorable outcome (P=0.001, FIG. 9B), validating the prognostic value of the PY-STAT3 signature. Within Cluster 4, the BCL6-negative cases had the most adverse survival (P=0.089, FIG. 9C). Survival distributions of Clusters 1 and 3 also differ significantly (P=0.022). Since this cohort of 233 cases has been previously analyzed using a Survival Predictor Score, a composite of the GCB and the two stromal signatures,¹⁴ distribution of this score among the 4 clusters was tested using gene set enrichment analysis (GSEA). The average Survival Predictor Score steadily increased from Cluster 1/2 to Cluster 3 and Cluster 4 (FIG. 9D). Since both Clusters 2 and 3 expressed Module A genes and yet only Cluster 3 cases were associated with inferior outcome, this result confirms the IHC-based finding that PY-STAT3 positivity predicted poor survival only when occurring in non-GCB-DLBCL patients.

To investigate the underlying biological basis for different survival response, a comparison was made of the relatively enriched genes in the ABC-DLBCL cases (62 in Cluster 3 and 25 in Cluster 4) using several previously curated gene-expression signatures. The Pan-T-cell signature¹⁴ was expressed at significantly higher levels in Cluster 3 than in Cluster 4 (T-test of median relative mRNA expression, P=0.007, FIG. 9E; and also Chi-square test of quartiles, P<0.001, Table 4), while Cluster 4 but not Cluster 3 had a significant enrichment of the B-cell proliferative signature²⁶ (T-test, P<0.001, FIG. 9F; and Chi-square test, P=0.007, Table 4). Since tumors in Cluster 4 showed very strong MUM1/IRF4 expression, these tumors might be blocked at the late stage of B-cell development featuring high level MUM1/IRF4. Indeed, when a plasmablastic GEP signature from multiple myeloma cells²⁷ was evaluated, significant enrichment was observed only in Cluster 4 (T-test, P<0.001, FIG. 9G; and Chi-square test, P<0.001, Table 4). The two previously recognized stromal signatures, Stromal-1 and Stromal-2, had similar enrichment patterns, i.e, both were highly expressed in Cluster 2 and strongly suppressed in Cluster 4 cases (FIG. 9A, bottom panel).

The Four PY-STAT3 Clusters Demonstrate Distinct Rituximab Sensitivity:

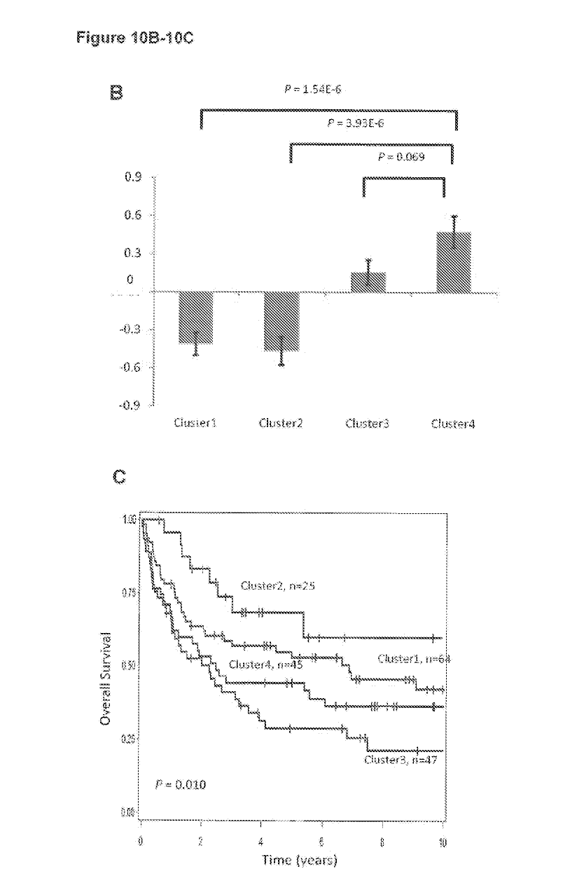

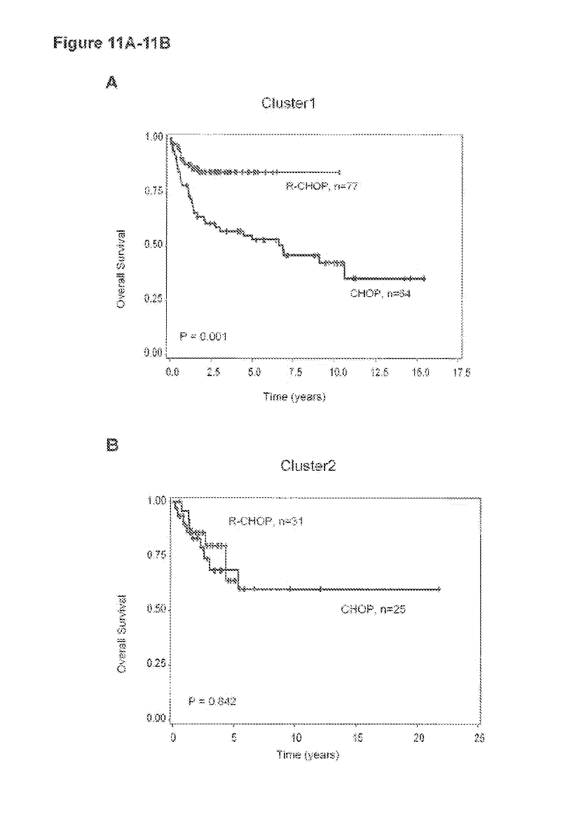

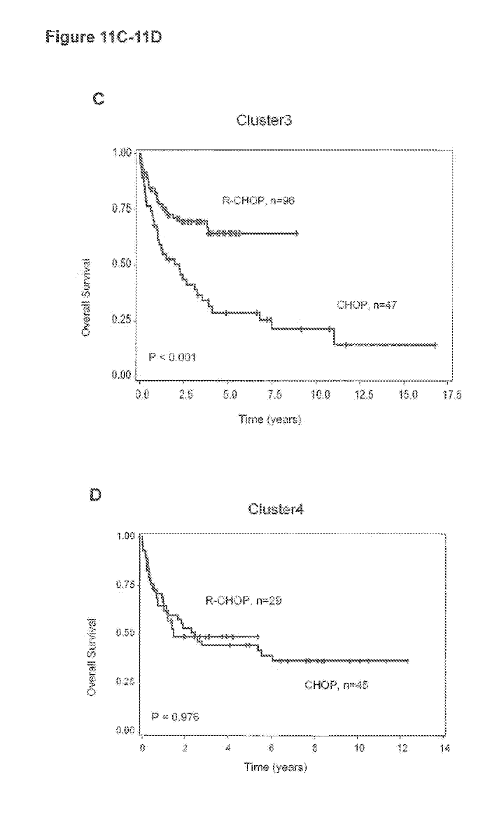

The 33-gene PY-STAT3 signature was also applied to a dataset of 181-case cohort treated with the CHOP therapy.¹⁴ Analyses showed that this 33-gene PY-STAT3 signature can similarly stratify this cohort into 4 subgroups with enrichment of Pan-T, proliferation, and plasmablastic signatures identical to those observed in the 233-case R-CHOP cohort (FIG. 10). There are, however, treatment-related differences in the survival outcome of individual clusters. While Cluster 2 and Cluster 3 identified the most and the least favorable subsets of patients in the CHOP cohort, respectively (FIG. 10C), these two subsets had similar OS in the R-CHOP cohort (FIG. 9B), suggesting different Clusters responded differently to the addition of rituximab to CHOP. This notion was confirmed when cluster-specific OS was compared between the R-CHOP and CHOP cohorts. Specifically, only patients in Cluster 1 and Cluster 3 but not those in Cluster 2 or Cluster 4 benefited significantly from the R-CHOP therapy (FIG. 11).

PLSR Model for the 4-Cluster DLBCL Data Classification:

An algorithm was developed to classify DLBCL cases into the 4 PY-STAT3 clusters using the 33-gene signature. This classifier is based on the partial least square regression (PLSR) model:

Y=Xb,

or

y_{pred}=b₀+b₁x₁+b₂x₂+ . . . +bₙxₙ

where y_{pred} is the predicted value and x₁, x₂, . . . xₙ is the expression value of each gene.

The PLSR model was applied for Module A and Module B genes, respectively. For Module A, the predicted covariate (Y) for Module A positive cases (Cluster 2 and 3) was set as 1, while predicted covariate (Y) for Module A negative cases (Cluster 1 and 4) was set as −1. The predicting covariates (X) were the expression values of the 25 genes in Module A. The same setting was applied for the Module B genes.

X and Y data were centered by their mean values before analysis, then PLSR was performed. The first PLS component was extracted from X and Y. For the Module A genes, the first PLS component stands for 25.5% of variance in X, and 55.0% of variance of Y. For the Module B genes, the first PLS component stands for 47.4% of variance in X, and 52.6 of variance of Y. The coefficient vector b for Module A and Module B genes is shown in Table 5.

For each DLBCL case, if ypred >0, it is the Module A/B positive case, otherwise, it is the Module A/B negative case. Predictive accuracy for Module A and B classifier is 90.6% and 85.4%, respectively. Then the predicted Cluster of each case is obtained based on the prediction result of Module A and B positivity. As shown in Table 6, total predictive accuracy is 76.8% (179/233) for the 233 DLBCL cases. The predictive accuracy for Cluster 1-4 is 71.4% (55/77), 51.6% (16/31), 86.5% (83/96), and 86.2% (25/29), respectively.

TABLE 1
Clinical and pathological characteristics of DLBCL patients
according to the PY-STAT3 expression.

| PY-STAT3 | |
|---|---|
| Expression | |
| Negative | Positive |
| (n = 116) | (n = 69) |

| | | | | |
|---|---|---|---|---|
| No. | % | No. | % | P-value |

Clinical characteristics

| | | |
|---|---|---|
| Age (years) | | |
| Median | 62.6 | 66.1 |
| Range | 19.6~87.2 | 23.6~89.2 |

| | | | | | |
|---|---|---|---|---|---|
| <60 | 58 | 50.0 | 27 | 39.1 | 0.200 |
| ≧60 | 58 | 50.0 | 42 | 60.9 | |
| Gender | | | | | |
| Male | 67 | 57.8 | 35 | 49.3 | 0.438 |
| Female | 49 | 42.2 | 34 | 50.7 | |
| KS | | | | | |
| Performance | | | | | |
| >70 | 98 | 87.5 | 58 | 86.6 | 1 |
| ≦70 | 14 | 12.5 | 9 | 13.4 | |
| Stage | | | | | |
| I to II | 60 | 53.6 | 27 | 41.5 | 0.165 |
| III to IV | 52 | 46.4 | 38 | 58.5 | |
| Extranodal | | | | | |
| sites | | | | | |
| <2 | 99 | 87.6 | 55 | 82.1 | 0.424 |
| ≧2 | 14 | 12.4 | 12 | 17.9 | |
| Serum LDH | | | | | |
| Normal | 71 | 64.0 | 33 | 54.1 | 0.269 |
| Elevated | 40 | 36.0 | 28 | 45.9 | |
| IPI risk group | | | | | |
| Low (0~2) | 88 | 80.0 | 41 | 67.2 | 0.094 |
| High (3~5) | 22 | 20.0 | 20 | 32.8 | |

Pathological characteristics

| | | | | | |
|---|---|---|---|---|---|
| Subtypes | | | | | |
| GCB | 67 | 57.8 | 29 | 40.0 | 0.030 |
| non-GCB/ABC | 41 | 35.3 | 37 | 53.6 | |
| NC | 8 | 6.9 | 3 | 6.4 | |
| BCL6 | | | | | |
| expression | | | | | |
| Negative | 47 | 42.7 | 29 | 43.3 | 0.920 |
| Positive | 63 | 57.3 | 38 | 56.7 | |
| MUM1/IRF4 | | | | | |
| expression | | | | | |
| Negative | 53 | 48.2 | 21 | 31.3 | 0.041 |
| Positive | 57 | 51.8 | 46 | 68.7 | |

Abbreviations:
Abbreviations:
KS, Karnofsky score;
LDH, lactate dehydrogenase;
IPI, international prognostic index.

TABLE 2
Patient characteristics according to STAT3 mRNA levels.

| | STAT3 mRNA Expression | | | | | | |
|---|---|---|---|---|---|---|---|
| | Low | | Intermediate | | High | | |
| | (n = 37) | | (n = 156) | | (n = 29) | | |
| Characteristic | No. | % | No. | % | No. | % | P-value |
| Age (years) | | | | | | | |
| Median | 60.7 | | 60.9 | | 62.3 | | |
| Range | 35.6~84.8 | | 30.3~85.8 | | 16.7~92.0 | | |
| <60 | 18 | 48.6 | 78 | 50.0 | 13 | 44.8 | 0.876 |
| ≧60 | 19 | 52.4 | 78 | 50.0 | 16 | 55.2 | |
| Sex | | | | | | | |
| Male | 17 | 45.9 | 92 | 59.0 | 20 | 69.0 | 0.157 |
| Female | 20 | 54.1 | 64 | 41.0 | 9 | 31.0 | |
| KS Performance | | | | | | | |
| >70 | 30 | 81.1 | 121 | 77.6 | 21 | 72.4 | 0.704 |
| ≦70 | 7 | 18.9 | 35 | 22.4 | 8 | 27.6 | |
| Stage | | | | | | | |
| I to II | 20 | 54.1 | 74 | 47.4 | 13 | 44.8 | 0.713 |
| III to IV | 17 | 45.9 | 82 | 52.6 | 16 | 55.2 | |
| Extranodal sites | | | | | | | |
| <2 | 28 | 75.7 | 138 | 88.5 | 26 | 89.7 | 0.107 |
| ≧2 | 9 | 24.3 | 18 | 11.5 | 3 | 10.3 | |
| Serum LDH | | | | | | | |
| Normal | 21 | 56.8 | 97 | 62.2 | 15 | 51.7 | 0.523 |
| Elevated | 16 | 43.2 | 59 | 37.8 | 14 | 48.3 | |
| IPI risk group | | | | | | | |
| Low (0~2) | 26 | 70.3 | 113 | 72.4 | 20 | 69.0 | 0.912 |
| High (3~5) | 11 | 29.7 | 43 | 27.6 | 9 | 31.0 | |
| DLBCL subtype | | | | | | | |
| GCB | 26 | 70.3 | 73 | 46.8 | 3 | 10.3 | <0.001 |
| ABC | 10 | 27.0 | 55 | 35.3 | 24 | 82.8 | |
| NC | 1 | | 28 | | 2 | | |
| BCL6 | | | | | | | |
| Negative | 13 | 59.1 | 70 | 55.6 | 15 | 62.5 | 0.801 |
| Positive | 9 | 40.9 | 56 | 44.4 | 9 | 37.5 | |
| MUM1/IRF4 | | | | | | | |
| Negative | 10 | 45.5 | 58 | 45.7 | 3 | 13.6 | 0.007 |
| Positive | 12 | 54.5 | 69 | 54.3 | 22 | 86.4 | |
| PY-STAT3 (IHC) | | | | | | | |
| Negative | 9 | 81.8 | 58 | 84.1 | 6 | 33.3 | <0.001 |
| Positive | 2 | 18.2 | 11 | 15.9 | 12 | 66.7 | |

NOTE.
DLBCL cases are classified by STAT3 mRNA expression into high (>mean + S.D.), low (<mean − S.D.), and intermediated (the rest of the cases) groups.

TABLE 3
Multivariate hazard analysis of DLBCL patients by IPI
score, GCB/non-GCB subclassification and PY-STAT3.

| Analysis of Survival | HR | 95% CI | P-value |
|---|---|---|---|
| OS | | | |
| Non-GCB/ABC vs GCB | 1.18 | 0.67-2.07 | 0.559 |
| IPI 3-5 vs 0-2 | 2.40 | 1.35-4.27 | 0.003 |
| PY-STAT3 High vs Low | 1.79 | 1.02-3.14 | 0.041 |
| EFS | | | |
| Non-GCB/ABC vs GCB | 1.45 | 0.88-2.39 | 0.148 |
| IPI 3-5 vs 0-2 | 1.79 | 1.06-30.1 | 0.029 |
| PY-STAT3 High vs Low | 1.79 | 1.09-2.95 | 0.022 |

Abbreviations: OS, overall survival; EFS, event-free survival; HR, hazard ratio; CI, confidence interval; IPI, international prognostic index.

TABLE 4
Distribution of ABC-DLBCL cases in Cluster 3 (n = 62)
and Cluster 4 (n = 25) according to the relative mRNA
expression of Pan-T-Cell, proliferative, and plasmablastic signatures.

| | Quartiles | | | | |
|---|---|---|---|---|---|
| Signatures | Quartile 1 | Quartile 2 | Quartile 3 | Quartile 4 | P-value |
| Pan-T-cell | | | | | |
| Median | −1.20 | −0.30 | 0.35 | 1.14 | |
| Range | −2.09~−0.64 | −0.61~0.06 | 0.07~0.66 | 0.68~2.06 | |
| No. in | 8 | 14 | 20 | 20 | <0.001 |
| Cluster 3 | | | | | |
| No. in | 14 | 8 | 2 | 1 | |
| Cluster 4 | | | | | |
| Prolif- | | | | | |
| erative | | | | | |
| Median | −0.64 | −0.11 | 0.04 | 0.60 | |
| Range | −1.01~−0.40 | −0.38~0.02 | 0.04~0.40 | 0.41~1.30 | |
| No. in | 21 | 16 | 15 | 10 | 0.007 |
| Cluster 3 | | | | | |
| No. in | 1 | 6 | 7 | 11 | |
| Cluster 4 | | | | | |
| Plas- | | | | | |
| mablastic | | | | | |
| Median | −0.30 | −0.03 | 0.07 | 0.29 | |
| Range | −0.77~0.18 | −0.16~0.0 | 0.01~0.19 | 0.20~0.49 | |
| No. in | 23 | 20 | 16 | 10 | <0.001 |
| Cluster 3 | | | | | |
| No. in | 1 | 3 | 7 | 13 | |
| Cluster 4 | | | | | |

The median relative mRNA expression of each case was calculated and sorted in ascending order. Chi-square test was used to evaluate the distribution of cases in Cluster 3 vs Cluster 4 among the four quartiles.

TABLE 5
Coefficients of the first PLS component
for Module A and Module B genes.

| Module A | | | Module B | | |
|---|---|---|---|---|---|
| Genes (X_{A}) | Coefficients (b) | | Genes (X_{B}) | Coefficients (b) | |
| | b0 | −11.0066 | | b0 | −9.0456 |
| BATF | b1 | 0.0298 | BTLA | b1 | 0.1318 |
| CAPN2 | b2 | 0.0600 | C13orf18 | b2 | 0.1682 |
| CCND2 | b3 | 0.0472 | CFLAR | b3 | 0.1400 |
| CD2 | b4 | 0.0901 | EVI2A | b4 | 0.0394 |
| CMTM3 | b5 | 0.0318 | HIST2H2AA3 | b5 | 0.0939 |
| DYNLT1 | b6 | 0.0280 | IL16 | b6 | 0.1029 |
| ELL2 | b7 | 0.0419 | IL2RA | b7 | 0.0606 |
| GALNT1 | b8 | 0.0217 | PTGER4 | b8 | 0.0986 |
| GCA | b9 | 0.0216 | | | |
| GMFG | b10 | 0.0241 | | | |
| GYG1 | b11 | 0.0206 | | | |
| GZMB | b12 | 0.0817 | | | |
| MAN1A1 | b13 | 0.0266 | | | |
| MEX3D | b14 | 0.0069 | | | |
| MT1X | b15 | 0.0499 | | | |
| PERP | b16 | 0.0454 | | | |
| PLAGL1 | b17 | 0.0248 | | | |
| PRF1 | b18 | 0.0795 | | | |
| RAB27A | b19 | 0.0454 | | | |
| S100A6 | b20 | 0.0373 | | | |
| SERPINB1 | b21 | 0.0243 | | | |
| TTC39C | b22 | 0.0480 | | | |
| XK | b23 | 0.0171 | | | |
| ZBED2 | b24 | 0.0833 | | | |
| ZNRF1 | b25 | 0.0700 | | | |

TABLE 6
PLSR predictive result for the 233 DLBCL cases.

| | Module | Module A | Module A | Module | Module B | Module B | | Predicted | Predict |
|---|---|---|---|---|---|---|---|---|---|
| NAME | A Score | Pred-Value | Pred-Class | B Score | Pred-Value | Pred-Class | Cluster | Cluster | Right? |
| GSM275076 | 1 | 0.435 | 1 | 1 | 0.665 | 1 | 3 | 3 | 1 |
| GSM275077 | 1 | 0.462 | 1 | 1 | 0.567 | 1 | 3 | 3 | 1 |
| GSM275078 | 1 | 0.550 | 1 | 1 | 0.264 | 1 | 3 | 3 | 1 |
| GSM275079 | 1 | 0.796 | 1 | 1 | 0.679 | 1 | 3 | 3 | 1 |
| GSM275080 | 1 | 0.783 | 1 | 1 | 0.914 | 1 | 3 | 3 | 1 |
| GSM275081 | 1 | 0.826 | 1 | 1 | 0.513 | 1 | 3 | 3 | 1 |
| GSM275082 | 1 | 0.273 | 1 | 1 | 0.414 | 1 | 3 | 3 | 1 |
| GSM275083 | 1 | 0.190 | 1 | −1 | −0.197 | −1 | 2 | 2 | 1 |
| GSM275084 | −1 | −1.763 | −1 | −1 | −0.834 | −1 | 1 | 1 | 1 |
| GSM275085 | −1 | 0.393 | 1 | −1 | −0.364 | −1 | 1 | 2 | 0 |
| GSM275086 | −1 | −0.865 | −1 | 1 | 1.363 | 1 | 4 | 4 | 1 |
| GSM275087 | −1 | −2.756 | −1 | −1 | −0.695 | −1 | 1 | 1 | 1 |
| GSM275088 | 1 | 0.310 | 1 | 1 | 0.331 | 1 | 3 | 3 | 1 |
| GSM275089 | −1 | −0.500 | −1 | −1 | −0.203 | −1 | 1 | 1 | 1 |
| GSM275090 | 1 | 0.790 | 1 | 1 | 0.967 | 1 | 3 | 3 | 1 |
| GSM275091 | 1 | 0.246 | 1 | 1 | 0.403 | 1 | 3 | 3 | 1 |
| GSM275092 | −1 | 0.216 | 1 | −1 | −0.401 | −1 | 1 | 2 | 0 |
| GSM275093 | 1 | 0.707 | 1 | 1 | 0.345 | 1 | 3 | 3 | 1 |
| GSM275094 | −1 | −0.265 | −1 | −1 | −0.933 | −1 | 1 | 1 | 1 |
| GSM275095 | 1 | 1.000 | 1 | 1 | 0.097 | 1 | 3 | 3 | 1 |
| GSM275096 | −1 | −0.587 | −1 | −1 | −0.631 | −1 | 1 | 1 | 1 |
| GSM275097 | −1 | −0.352 | −1 | 1 | 0.326 | 1 | 4 | 4 | 1 |
| GSM275098 | −1 | −0.917 | −1 | −1 | −0.441 | −1 | 1 | 1 | 1 |
| GSM275099 | −1 | −0.811 | −1 | −1 | −0.272 | −1 | 1 | 1 | 1 |
| GSM275100 | −1 | −0.216 | −1 | 1 | 1.294 | 1 | 4 | 4 | 1 |
| GSM275101 | −1 | −0.274 | −1 | −1 | 0.541 | 1 | 1 | 4 | 0 |
| GSM275102 | 1 | 0.505 | 1 | 1 | −0.001 | −1 | 3 | 2 | 0 |
| GSM275103 | −1 | 0.112 | 1 | −1 | −0.508 | −1 | 1 | 2 | 0 |
| GSM275104 | 1 | 0.162 | 1 | 1 | 0.141 | 1 | 3 | 3 | 1 |
| GSM275105 | 1 | 1.208 | 1 | 1 | −0.052 | −1 | 3 | 2 | 0 |
| GSM275106 | −1 | −0.187 | −1 | −1 | −0.987 | −1 | 1 | 1 | 1 |
| GSM275107 | −1 | −0.368 | −1 | −1 | −0.914 | −1 | 1 | 1 | 1 |
| GSM275108 | −1 | −1.459 | −1 | −1 | −1.542 | −1 | 1 | 1 | 1 |
| GSM275109 | −1 | −0.099 | −1 | 1 | 0.417 | 1 | 4 | 4 | 1 |
| GSM275110 | 1 | 0.415 | 1 | −1 | −0.762 | −1 | 2 | 2 | 1 |
| GSM275111 | 1 | 0.632 | 1 | 1 | 0.453 | 1 | 3 | 3 | 1 |
| GSM275112 | 1 | 0.472 | 1 | −1 | 0.496 | 1 | 2 | 3 | 0 |
| GSM275113 | 1 | 0.618 | 1 | −1 | −0.987 | −1 | 2 | 2 | 1 |
| GSM275114 | −1 | −0.262 | −1 | −1 | −0.645 | −1 | 1 | 1 | 1 |
| GSM275115 | 1 | 0.141 | 1 | 1 | 1.184 | 1 | 3 | 3 | 1 |
| GSM275116 | 1 | 0.668 | 1 | 1 | −0.211 | −1 | 3 | 2 | 0 |
| GSM275117 | −1 | −0.394 | −1 | −1 | −1.141 | −1 | 1 | 1 | 1 |
| GSM275118 | −1 | −0.272 | −1 | −1 | −1.306 | −1 | 1 | 1 | 1 |
| GSM275119 | −1 | −0.168 | −1 | 1 | 0.977 | 1 | 4 | 4 | 1 |
| GSM275120 | 1 | 0.152 | 1 | 1 | 0.859 | 1 | 3 | 3 | 1 |
| GSM275121 | 1 | 0.655 | 1 | 1 | 0.485 | 1 | 3 | 3 | 1 |
| GSM275122 | 1 | 0.568 | 1 | 1 | 0.149 | 1 | 3 | 3 | 1 |
| GSM275123 | 1 | 0.730 | 1 | 1 | −0.366 | −1 | 3 | 2 | 0 |
| GSM275124 | −1 | −0.454 | −1 | −1 | 0.009 | 1 | 1 | 4 | 0 |
| GSM275125 | −1 | 0.338 | 1 | 1 | 0.454 | 1 | 4 | 3 | 0 |
| GSM275126 | −1 | −0.403 | −1 | −1 | −1.456 | −1 | 1 | 1 | 1 |
| GSM275127 | −1 | −0.470 | −1 | −1 | −1.393 | −1 | 1 | 1 | 1 |
| GSM275128 | 1 | 0.738 | 1 | 1 | 1.092 | 1 | 3 | 3 | 1 |
| GSM275129 | −1 | 0.004 | 1 | −1 | −1.253 | −1 | 1 | 2 | 0 |
| GSM275130 | −1 | −0.759 | −1 | 1 | 0.919 | 1 | 4 | 4 | 1 |
| GSM275131 | 1 | 0.265 | 1 | 1 | 0.077 | 1 | 3 | 3 | 1 |
| GSM275132 | 1 | 1.283 | 1 | 1 | 0.848 | 1 | 3 | 3 | 1 |
| GSM275133 | −1 | −0.454 | −1 | −1 | −0.557 | −1 | 1 | 1 | 1 |
| GSM275134 | −1 | 0.215 | 1 | −1 | −0.381 | −1 | 1 | 2 | 0 |
| GSM275135 | 1 | 1.575 | 1 | 1 | 1.000 | 1 | 3 | 3 | 1 |
| GSM275136 | 1 | 1.253 | 1 | 1 | 1.090 | 1 | 3 | 3 | 1 |
| GSM275137 | −1 | −0.562 | −1 | −1 | −0.567 | −1 | 1 | 1 | 1 |
| GSM275138 | 1 | 0.613 | 1 | −1 | −0.240 | −1 | 2 | 2 | 1 |
| GSM275139 | 1 | 0.277 | 1 | 1 | 1.635 | 1 | 3 | 3 | 1 |
| GSM275140 | 1 | 0.534 | 1 | 1 | 0.955 | 1 | 3 | 3 | 1 |
| GSM275141 | −1 | −0.343 | −1 | 1 | 0.646 | 1 | 4 | 4 | 1 |
| GSM275142 | −1 | −0.013 | −1 | 1 | 0.662 | 1 | 4 | 4 | 1 |
| GSM275143 | −1 | −0.639 | −1 | −1 | 0.464 | 1 | 1 | 4 | 0 |
| GSM275144 | −1 | −1.429 | −1 | −1 | 0.062 | 1 | 1 | 4 | 0 |
| GSM275145 | 1 | 0.194 | 1 | −1 | 0.313 | 1 | 2 | 3 | 0 |
| GSM275146 | −1 | −1.299 | −1 | −1 | −0.440 | −1 | 1 | 1 | 1 |
| GSM275147 | 1 | 0.202 | 1 | 1 | 0.453 | 1 | 3 | 3 | 1 |
| GSM275148 | −1 | −0.186 | −1 | −1 | −0.166 | −1 | 1 | 1 | 1 |
| GSM275149 | 1 | 1.097 | 1 | 1 | −0.316 | −1 | 3 | 2 | 0 |
| GSM275150 | 1 | 0.502 | 1 | 1 | 0.673 | 1 | 3 | 3 | 1 |
| GSM275151 | 1 | 0.171 | 1 | 1 | 0.692 | 1 | 3 | 3 | 1 |
| GSM275152 | −1 | 0.236 | 1 | −1 | 0.210 | 1 | 1 | 3 | 0 |
| GSM275153 | −1 | −1.254 | −1 | −1 | −1.694 | −1 | 1 | 1 | 1 |
| GSM275154 | −1 | −0.463 | −1 | −1 | −0.767 | −1 | 1 | 1 | 1 |
| GSM275155 | −1 | −0.705 | −1 | 1 | 1.212 | 1 | 4 | 4 | 1 |
| GSM275156 | −1 | 0.191 | 1 | −1 | −0.955 | −1 | 1 | 2 | 0 |
| GSM275157 | −1 | −0.609 | −1 | 1 | 0.549 | 1 | 4 | 4 | 1 |
| GSM275158 | −1 | −0.361 | −1 | −1 | −0.177 | −1 | 1 | 1 | 1 |
| GSM275159 | 1 | 0.775 | 1 | 1 | 0.945 | 1 | 3 | 3 | 1 |
| GSM275160 | 1 | 0.342 | 1 | 1 | 1.040 | 1 | 3 | 3 | 1 |
| GSM275161 | −1 | −0.732 | −1 | 1 | 0.635 | 1 | 4 | 4 | 1 |
| GSM275162 | 1 | 0.466 | 1 | 1 | 0.175 | 1 | 3 | 3 | 1 |
| GSM275163 | 1 | 0.415 | 1 | −1 | 0.244 | 1 | 2 | 3 | 0 |
| GSM275164 | −1 | −0.527 | −1 | −1 | −1.256 | −1 | 1 | 1 | 1 |
| GSM275165 | 1 | 1.144 | 1 | 1 | 0.564 | 1 | 3 | 3 | 1 |
| GSM275166 | −1 | −0.896 | −1 | −1 | −0.245 | −1 | 1 | 1 | 1 |
| GSM275167 | 1 | 0.702 | 1 | 1 | 0.063 | 1 | 1 | 3 | 0 |
| GSM275168 | 1 | 0.442 | 1 | 1 | 0.359 | 1 | 3 | 3 | 1 |
| GSM275169 | 1 | 0.390 | 1 | 1 | 0.785 | 1 | 3 | 3 | 1 |
| GSM275170 | 1 | 0.724 | 1 | 1 | 0.197 | 1 | 3 | 3 | 1 |
| GSM275171 | −1 | −0.069 | −1 | −1 | 0.116 | 1 | 1 | 4 | 0 |
| GSM275172 | 1 | 0.443 | 1 | 1 | 1.647 | 1 | 3 | 3 | 1 |
| GSM275173 | 1 | 1.227 | 1 | 1 | 0.236 | 1 | 3 | 3 | 1 |
| GSM275174 | −1 | −1.139 | −1 | 1 | 0.924 | 1 | 4 | 4 | 1 |
| GSM275175 | −1 | −1.506 | −1 | −1 | −0.831 | −1 | 1 | 1 | 1 |
| GSM275176 | −1 | −0.255 | −1 | 1 | 0.711 | 1 | 4 | 4 | 1 |
| GSM275177 | 1 | 0.420 | 1 | 1 | 0.465 | 1 | 3 | 3 | 1 |
| GSM275178 | −1 | −0.311 | −1 | −1 | −1.324 | −1 | 1 | 1 | 1 |
| GSM275179 | −1 | −0.331 | −1 | 1 | 1.124 | 1 | 4 | 4 | 1 |
| GSM275180 | −1 | −0.015 | −1 | −1 | −0.837 | −1 | 1 | 1 | 1 |
| GSM275181 | 1 | 0.780 | 1 | 1 | 0.612 | 1 | 3 | 3 | 1 |
| GSM275182 | 1 | 0.452 | 1 | −1 | 0.151 | 1 | 2 | 3 | 0 |
| GSM275183 | 1 | 1.106 | 1 | 1 | 0.457 | 1 | 3 | 3 | 1 |
| GSM275184 | −1 | −1.174 | −1 | −1 | −1.359 | −1 | 1 | 1 | 1 |
| GSM275185 | 1 | 1.313 | 1 | 1 | 0.695 | 1 | 3 | 3 | 1 |
| GSM275186 | 1 | 0.500 | 1 | −1 | 0.005 | 1 | 2 | 3 | 0 |
| GSM275187 | −1 | −0.108 | −1 | −1 | −0.294 | −1 | 1 | 1 | 1 |
| GSM275188 | 1 | 1.153 | 1 | 1 | 0.728 | 1 | 3 | 3 | 1 |
| GSM275189 | 1 | 0.649 | 1 | 1 | 0.912 | 1 | 3 | 3 | 1 |
| GSM275190 | 1 | 0.658 | 1 | −1 | −0.448 | −1 | 2 | 2 | 1 |
| GSM275191 | −1 | −0.943 | −1 | 1 | 0.813 | 1 | 4 | 4 | 1 |
| GSM275192 | 1 | 1.167 | 1 | 1 | 1.075 | 1 | 3 | 3 | 1 |
| GSM275193 | 1 | 0.041 | 1 | −1 | −0.638 | −1 | 2 | 2 | 1 |
| GSM275194 | −1 | −1.258 | −1 | −1 | −0.467 | −1 | 1 | 1 | 1 |
| GSM275195 | 1 | 0.764 | 1 | 1 | 0.238 | 1 | 3 | 3 | 1 |
| GSM275196 | 1 | 0.674 | 1 | 1 | 1.042 | 1 | 3 | 3 | 1 |
| GSM275197 | −1 | −0.160 | −1 | −1 | −1.006 | −1 | 1 | 1 | 1 |
| GSM275198 | 1 | 1.244 | 1 | 1 | 0.905 | 1 | 3 | 3 | 1 |
| GSM275199 | 1 | 0.388 | 1 | 1 | 0.304 | 1 | 3 | 3 | 1 |
| GSM275200 | 1 | 1.127 | 1 | 1 | 0.334 | 1 | 3 | 3 | 1 |
| GSM275201 | −1 | −0.194 | −1 | −1 | −0.712 | −1 | 1 | 1 | 1 |
| GSM275202 | 1 | 0.738 | 1 | −1 | 0.417 | 1 | 2 | 3 | 0 |
| GSM275203 | 1 | 0.728 | 1 | 1 | 1.459 | 1 | 3 | 3 | 1 |
| GSM275204 | 1 | 0.764 | 1 | 1 | 0.530 | 1 | 3 | 3 | 1 |
| GSM275205 | 1 | 0.110 | 1 | −1 | 0.144 | 1 | 2 | 3 | 0 |
| GSM275206 | 1 | 0.828 | 1 | 1 | 1.584 | 1 | 3 | 3 | 1 |
| GSM275207 | 1 | 0.562 | 1 | 1 | 1.961 | 1 | 3 | 3 | 1 |
| GSM275208 | 1 | −0.018 | −1 | 1 | 0.136 | 1 | 1 | 4 | 0 |
| GSM275209 | −1 | −0.519 | −1 | −1 | −0.347 | −1 | 1 | 1 | 1 |
| GSM275210 | 1 | 1.666 | 1 | 1 | −0.116 | −1 | 3 | 2 | 0 |
| GSM275211 | −1 | −0.072 | −1 | −1 | −0.352 | −1 | 1 | 1 | 1 |
| GSM275212 | −1 | −0.214 | −1 | −1 | −0.770 | −1 | 1 | 1 | 1 |
| GSM275213 | −1 | −1.036 | −1 | −1 | −0.403 | −1 | 1 | 1 | 1 |
| GSM275214 | 1 | 1.093 | 1 | 1 | −0.490 | −1 | 3 | 2 | 0 |
| GSM275215 | 1 | 0.721 | 1 | −1 | −0.074 | −1 | 2 | 2 | 1 |
| GSM275216 | 1 | 0.237 | 1 | 1 | 1.054 | 1 | 3 | 3 | 1 |
| GSM275217 | −1 | −0.269 | −1 | 1 | 1.056 | 1 | 4 | 4 | 1 |
| GSM275218 | −1 | −0.620 | −1 | −1 | −0.464 | −1 | 1 | 1 | 1 |
| GSM275219 | 1 | 0.328 | 1 | 1 | 0.056 | 1 | 3 | 3 | 1 |
| GSM275220 | −1 | −0.546 | −1 | −1 | −0.367 | −1 | 1 | 1 | 1 |
| GSM275221 | −1 | −0.774 | −1 | 1 | 1.360 | 1 | 4 | 4 | 1 |
| GSM275222 | −1 | −0.308 | −1 | −1 | −0.787 | −1 | 1 | 1 | 1 |
| GSM275223 | 1 | 1.796 | 1 | 1 | 0.301 | 1 | 3 | 3 | 1 |
| GSM275224 | 1 | 1.422 | 1 | −1 | −0.908 | −1 | 2 | 2 | 1 |
| GSM275225 | 1 | 0.494 | 1 | 1 | 0.354 | 1 | 3 | 3 | 1 |
| GSM275226 | 1 | 0.093 | 1 | 1 | 0.716 | 1 | 3 | 3 | 1 |
| GSM275227 | −1 | −0.441 | −1 | −1 | −0.061 | −1 | 1 | 1 | 1 |
| GSM275228 | −1 | −0.217 | −1 | −1 | −0.845 | −1 | 1 | 1 | 1 |
| GSM275229 | −1 | 0.134 | 1 | 1 | 0.861 | 1 | 4 | 3 | 0 |
| GSM275230 | −1 | −0.920 | −1 | −1 | −0.568 | −1 | 1 | 1 | 1 |
| GSM275231 | 1 | 0.775 | 1 | 1 | 0.167 | 1 | 3 | 3 | 1 |
| GSM275232 | 1 | 0.128 | 1 | −1 | −0.693 | −1 | 2 | 2 | 1 |
| GSM275233 | −1 | 0.264 | 1 | −1 | −0.702 | −1 | 1 | 2 | 0 |
| GSM275234 | −1 | −0.759 | −1 | −1 | −1.183 | −1 | 1 | 1 | 1 |
| GSM275235 | 1 | 0.618 | 1 | 1 | 0.639 | 1 | 3 | 3 | 1 |
| GSM275236 | 1 | 0.709 | 1 | 1 | 0.583 | 1 | 3 | 3 | 1 |
| GSM275237 | 1 | 0.673 | 1 | 1 | 0.845 | 1 | 3 | 3 | 1 |
| GSM275238 | 1 | 0.404 | 1 | 1 | 0.219 | 1 | 3 | 3 | 1 |
| GSM275239 | −1 | −0.389 | −1 | −1 | 0.121 | 1 | 1 | 4 | 0 |
| GSM275240 | 1 | 0.296 | 1 | 1 | 0.304 | 1 | 3 | 3 | 1 |
| GSM275241 | −1 | −0.793 | −1 | 1 | 1.031 | 1 | 4 | 4 | 1 |
| GSM275242 | 1 | 0.571 | 1 | 1 | 1.291 | 1 | 3 | 3 | 1 |
| GSM275243 | −1 | −0.586 | −1 | −1 | 0.078 | 1 | 1 | 4 | 0 |
| GSM275244 | 1 | 1.220 | 1 | 1 | 0.325 | 1 | 3 | 3 | 1 |
| GSM275245 | 1 | 1.166 | 1 | 1 | 0.308 | 1 | 3 | 3 | 1 |
| GSM275246 | 1 | 0.491 | 1 | 1 | −0.132 | −1 | 3 | 2 | 0 |
| GSM275247 | 1 | 0.851 | 1 | 1 | 0.387 | 1 | 3 | 3 | 1 |
| GSM275248 | 1 | 0.294 | 1 | −1 | 0.200 | 1 | 2 | 3 | 0 |
| GSM275249 | 1 | 0.808 | 1 | 1 | 0.509 | 1 | 3 | 3 | 1 |
| GSM275250 | 1 | −0.354 | −1 | −1 | 0.077 | 1 | 2 | 4 | 0 |
| GSM275251 | −1 | −0.250 | −1 | −1 | −0.380 | −1 | 1 | 1 | 1 |
| GSM275252 | −1 | −0.620 | −1 | −1 | −0.006 | −1 | 1 | 1 | 1 |
| GSM275253 | 1 | −0.042 | −1 | −1 | −0.065 | −1 | 2 | 1 | 0 |
| GSM275254 | 1 | 1.521 | 1 | 1 | 0.020 | 1 | 3 | 3 | 1 |
| GSM275255 | 1 | 0.794 | 1 | −1 | −0.056 | −1 | 2 | 2 | 1 |
| GSM275256 | 1 | 0.368 | 1 | 1 | 0.774 | 1 | 3 | 3 | 1 |
| GSM275257 | 1 | 0.628 | 1 | 1 | −0.200 | −1 | 3 | 2 | 0 |
| GSM275258 | −1 | −0.606 | −1 | −1 | −0.428 | −1 | 1 | 1 | 1 |
| GSM275259 | 1 | −1.073 | −1 | −1 | −1.084 | −1 | 2 | 1 | 0 |
| GSM275260 | 1 | 0.431 | 1 | −1 | −0.337 | −1 | 2 | 2 | 1 |
| GSM275261 | 1 | 0.582 | 1 | 1 | 0.868 | 1 | 3 | 3 | 1 |
| GSM275262 | 1 | 0.817 | 1 | −1 | −0.582 | −1 | 2 | 2 | 1 |
| GSM275263 | −1 | −0.204 | −1 | −1 | −0.140 | −1 | 1 | 1 | 1 |
| GSM275264 | 1 | −0.176 | −1 | −1 | −1.069 | −1 | 2 | 1 | 0 |
| GSM275265 | 1 | −1.104 | −1 | −1 | −1.453 | −1 | 2 | 1 | 0 |
| GSM275266 | −1 | −1.790 | −1 | −1 | 0.062 | 1 | 1 | 4 | 0 |
| GSM275267 | 1 | 0.477 | 1 | 1 | 0.388 | 1 | 3 | 3 | 1 |
| GSM275268 | −1 | 0.032 | 1 | −1 | −0.436 | −1 | 1 | 2 | 0 |
| GSM275269 | 1 | −0.025 | −1 | 1 | 0.188 | 1 | 3 | 4 | 0 |
| GSM275270 | −1 | −0.346 | −1 | −1 | −0.054 | −1 | 1 | 1 | 1 |
| GSM275271 | −1 | −1.071 | −1 | 1 | 0.103 | 1 | 4 | 4 | 1 |
| GSM275272 | 1 | 0.365 | 1 | 1 | 0.958 | 1 | 3 | 3 | 1 |
| GSM275273 | −1 | −1.544 | −1 | −1 | −1.750 | −1 | 1 | 1 | 1 |
| GSM275274 | 1 | 0.597 | 1 | 1 | 0.038 | 1 | 3 | 3 | 1 |
| GSM275275 | −1 | −0.493 | −1 | 1 | 0.437 | 1 | 4 | 4 | 1 |
| GSM275276 | 1 | 0.139 | 1 | −1 | −0.412 | −1 | 2 | 2 | 1 |
| GSM275277 | 1 | 0.630 | 1 | 1 | 1.200 | 1 | 3 | 3 | 1 |
| GSM275278 | 1 | 1.210 | 1 | 1 | 0.435 | 1 | 3 | 3 | 1 |
| GSM275279 | 1 | 0.317 | 1 | 1 | 0.820 | 1 | 3 | 3 | 1 |
| GSM275280 | 1 | 0.640 | 1 | −1 | 0.027 | 1 | 2 | 3 | 0 |
| GSM275281 | −1 | −0.374 | −1 | −1 | −0.466 | −1 | 1 | 1 | 1 |
| GSM275282 | 1 | 0.176 | 1 | −1 | −0.465 | −1 | 2 | 2 | 1 |
| GSM275283 | −1 | −1.205 | −1 | −1 | −0.201 | −1 | 1 | 1 | 1 |
| GSM275284 | −1 | −0.930 | −1 | −1 | −0.976 | −1 | 1 | 1 | 1 |
| GSM275285 | −1 | 0.155 | 1 | 1 | 0.302 | 1 | 4 | 3 | 0 |
| GSM275286 | −1 | −0.130 | −1 | 1 | 0.929 | 1 | 4 | 4 | 1 |
| GSM275287 | 1 | 0.545 | 1 | 1 | 0.248 | 1 | 3 | 3 | 1 |
| GSM275288 | 1 | 0.369 | 1 | −1 | −0.059 | −1 | 2 | 2 | 1 |
| GSM275289 | 1 | 0.837 | 1 | 1 | 0.128 | 1 | 3 | 3 | 1 |
| GSM275290 | −1 | 0.077 | 1 | 1 | 0.053 | 1 | 4 | 3 | 0 |
| GSM275291 | −1 | −0.189 | −1 | 1 | 1.183 | 1 | 4 | 4 | 1 |
| GSM275292 | 1 | 0.101 | 1 | 1 | −0.580 | −1 | 3 | 2 | 0 |
| GSM275293 | −1 | −0.701 | −1 | −1 | 0.212 | 1 | 1 | 4 | 0 |
| GSM275294 | 1 | −0.003 | −1 | −1 | 0.364 | 1 | 2 | 4 | 0 |
| GSM275295 | −1 | −0.047 | −1 | −1 | −0.804 | −1 | 1 | 1 | 1 |
| GSM275296 | −1 | 0.182 | 1 | −1 | −0.680 | −1 | 1 | 2 | 0 |
| GSM275297 | −1 | −0.249 | −1 | 1 | 1.102 | 1 | 4 | 4 | 1 |
| GSM275298 | 1 | 0.075 | 1 | 1 | −0.189 | −1 | 3 | 2 | 0 |
| GSM275299 | −1 | −1.315 | −1 | −1 | 0.400 | 1 | 1 | 4 | 0 |
| GSM275300 | 1 | 1.151 | 1 | 1 | 0.569 | 1 | 3 | 3 | 1 |
| GSM275301 | 1 | 2.137 | 1 | 1 | 0.032 | 1 | 3 | 3 | 1 |
| GSM275302 | 1 | 0.490 | 1 | 1 | 0.072 | 1 | 3 | 3 | 1 |
| GSM275303 | −1 | −0.614 | −1 | −1 | −0.711 | −1 | 1 | 1 | 1 |
| GSM275304 | 1 | 1.284 | 1 | 1 | −0.019 | −1 | 3 | 2 | 0 |
| GSM275305 | −1 | −0.688 | −1 | 1 | 1.099 | 1 | 4 | 4 | 1 |
| GSM275306 | −1 | −0.240 | −1 | −1 | −1.149 | −1 | 1 | 1 | 1 |
| GSM275307 | −1 | −0.097 | −1 | 1 | 0.148 | 1 | 4 | 4 | 1 |
| GSM275308 | 1 | 0.183 | 1 | −1 | −0.441 | −1 | 2 | 2 | 1 |

TABLE 7
The 11 genes in the STAT3 activation signature
for the prognosis of DLBCL patients.

| Probe set | Symbol | Name |
|---|---|---|
| 201413_at | HSD17B4 | Hydroxysteroid (17-beta) dehydrogenase 4 |
| 235536_at | RNF149 | Ring finger protein 149 |
| 238437_at | ZNF805 | Zinc finger protein 805 |
| 227176_at | SLC2A13 | Solute carrier family 2 (facilitated |
| | | glucose transporter), member 13 |
| 227633_at | RHEB | Ras homolog enriched in brain |
| 208581_x_at | MT1X | Metallothionein 1X |
| 235316_at | NAT8L | N-acetyltransferase 8-like (GCN5-related, |
| | | putative) |
| 218791_s_at | C15orf29 | Chromosome 15 open reading frame 29 |
| 238937_at | ZNF420 | Zinc finger protein 420 |
| 213159_at | PCNX | Pecanex homolog (Drosophila) |
| 203761_at | SLA | Src-like-adaptor |

TABLE 8
Distribution of IHC-defined PY-STAT3 positive cases
in the quartile subgroups based on mean expression
of the 11-gene STAT3 activation signature.

| | Quartiles | | | | |
|---|---|---|---|---|---|
| | Quartile 1 | Quartile 2 | Quartile 3 | Quartile 4 | P-value |
| All DLBCL | | | | | |
| cases (n = 98) | | | | | |
| PY-STAT3+ | 1 | 4 | 7 | 13 | 0.001 |
| PY-STAT3− | 24 | 21 | 15 | 13 | |
| ABC-DLBCL | | | | | |
| cases (n = 42) | | | | | |
| PY-STAT3+ | 2 | 2 | 2 | 8 | 0.005 |
| PY-STAT3− | 11 | 7 | 8 | 2 | |

Chi-square test was used to evaluate the distribution of PY-STAT3 positive versus negative cases among the four quartiles.

### Discussion

The studies described herein demonstrate that STAT3 activation has prognostic significance in patients with DLBCL, and its predictive power is much more significant when used in combination with other biomarkers, i.e. non-GCB immunophenotype for the R-CHOP regimen. This is believed to be the largest study to date demonstrating the prognostic significance of STAT3 activation in DLBCL patients treated with R-CHOP. In addition to providing strong and direct evidence that STAT3 activation is an independent prognostic biomarker in patients with DLBCL, the studies indicate that targeting STAT3 pathway may provide a novel therapeutic approach for patients with DLBCL.

The 33-gene PY-STAT3 GEP signature stratified R-CHOP treated DLBCL cases into 4 subgroups which have different immunophenotypes and, more importantly, exhibit marked differences in overall survival. The findings contradict the study by Lam et al. which reported no predictive value of a 23-gene STAT3 signature for DLBCL patients treated with CHOP regimen.⁹ For a direct comparison, the same cohort of patients was also analyzed using the 33-gene PY-STAT3 signature. The present GEP signature similarly stratified this cohort of DLBCL patients into 4 subgroups with different immunophenotypes and clinical outcomes (FIG. 10). The difference between the current study and that by Lam et al. might be attributed to the two GEP signatures which shared only one gene. Since the present 33-gene PY-STAT3 signature was constructed to predict the PY-STAT3 staining intensity in biopsy specimens, it reflects the status of Jak/STAT3 activation. In comparison, the 23-gene STAT3 signature correlated only with the presence of total STAT3 protein. Lam et al observed that among the STAT3-high subset of the ABC-DLBCL cases, 91% highly expressed total STAT3 protein yet only 57% were positive for PY-STAT3. This observation highlights the dis-concordance of these two markers in clinical specimens. The disparate prognosis significance between the two signatures suggests that it is the unique properties of an activated Jak/STAT3 pathway but not total STAT3 protein that is critical for the clinical outcome of patients with DLBCL.

An interesting property of the 33-gene PY-STAT3 signature is the fact that it contains two sub-Modules which were independently regulated across the entire phenotypic spectrum of DLBCL (FIG. 9A). There is a near perfect correlation between the GCB/ABC subgroups and the 4 PY-STAT3 clusters, i.e. Clusters 1 and 2 largely corresponded to the GCB cases while the ABC cases parted into Clusters 3 and 4. This is believed to be the first time a biomarker-based index can simultaneously subdivide both GCB- and ABC-DLBLC cases into prognostically relevant subgroups. Within normal GC, only those B cells located in the apical light zone are MUM1/IRF4 positive possibly because B cells can only interact with follicular dendritic cells and follicular T help cells from this location in order to activate the NF-κB signaling pathway.^{28,29} Since both Cluster 3 and 4 cases highly expressed MUM1/IRF4 and Cluster 4 further demonstrated plasmablastic features (FIG. 9G), 4 DLBCL clusters based on the PY-STAT3 signature are proposed to correspond to 4 different types of normal GC B cells: centroblasts/unactivated centrocytes (Cluster 1, BCL6+/MUM1−/STAT3−), partially activated centrocytes (Cluster 2, BCL6+/MUM1−/STAT3+/PY-STAT3^{low}), activated centrocytes/preplasmablasts (Cluster 3, BCL6^{low}/MUM1+/STAT3+/PY-STAT3^{high}) and plasmablasts (Cluster 4, BCL6−/MUM1+/STAT3^{low}/Blimp1+).^{8,28} Since MUM1/IRF4 is prominently expressed in ABC-DLBCL cases in general, notice is taken of a recent study showing that MUM1/IRF4 is required for upregulation of many STAT3 responsive genes during IL-21 treatment.³⁰ In support of a similar role for MUM1/IRF4 in DLBCL, the highest expression of Module A (Cluster 3) and activation of Module B (Cluster 3 and 4) predominantly occurred in MUM1/IRF4 positive, ABC-DLBCL cases (FIG. 9A). In comparison, in the Cluster 1 and 2 cases where MUM1/IRF4 was negative, there was a complete absence of Module B expression in both Clusters and only weak expression of Module A in Cluster 2.

Analysis of relative enrichment for the signatures also provided tantalizing clues regarding the tumor B cell-microenvironment interactions. Three types of microenvironment influences were evaluated in relation to the 4 PY-STAT3 clusters: a pan-T cell signature, and the two stromal signatures (FIG. 9A, bottom panels). Among the 4 clusters, the pan-T cell signature was significantly and selectively enriched in Clusters 2 and 3 which also expressed Module A genes. Given that in the normal GC microenvironment, a major STAT3 activating cytokine is IL-21 produced by follicular T help cells, the correlation between T cell enrichment and Module A suggests that in the DLBCL setting, tumor-associated STAT3 activation may still be dependent upon T-cell derived signals, such as IL-21. As discussed above, the fact that Module A genes were only weakly activated in Cluster 2 may be attributed, at least partially, to the absence of MUM1/IRF4.³⁰ Notice is also taken of the curious observation that Module B genes were negatively regulated by STAT3 in cell lines but showed the opposite trend within primary specimens such as the 30 training cases and those in Cluster 3. The reason for this discrepancy is currently unknown although non-B cell components in the tumor microenvironment may have altered the relationship between STAT3 and the expression of Module B genes. This hypothesis is in line with the observation that although many Cluster 4 cases had very little or no STAT3 mRNA, they continued to express Module B genes indicating STAT3-independent regulation (FIG. 9A, middle panel).

The biological insights uncovered in this study have direct implications for ongoing and future DLBCL clinical trials. The data showed that the cases least responsive to R-CHOP belonged to Cluster 4, the cluster showing plasmablastic features. Since Rituximab targets the CD20 molecule and normal plasma cells are typically CD20 negative, it is tempting to speculate that reduced CD20 expression may be responsible for the inferior outcome of Cluster 4 cases managed with R-CHOP. However, the analysis of CD20 mRNA expression does not support this theory (not shown). It cannot be ruled out that CD20 protein expression at the cell surface may be reduced in Cluster 4. From an experimental therapeutics perspective, the plasmablastic feature of Cluster 4 DLBCL suggests a new opportunity. Published mechanistic studies have shown that plasma cell differentiation is intrinsically linked to proteasomal overload and hence explains the exquisite sensitivity of multiple myeloma cells to proteasome inhibitor—containing therapies.^{31,32} In a recent phase I trial involving 49 DLBCL patients, it was found that the combination of DA-EPOCH plus bortezomib was efficacious only in ABC-DLBCL but not in GCB-DLBCL.³³ Based on these clinical observations and the results from this study, it is predicted that the ABC-DLBCL patients with plasmablastic tumors may benefit the most from the ongoing phase II trial of R-CHOP plus bortezomib.³⁴ Compared to Clusters 1 and 2, Cluster 3 patients also showed an adverse response to both CHOP and R-CHOP. Tumors in Cluster 3 features strong PY-STAT3 activation and a microenvironment highly enriched in T cells. Persistent STAT3 activation in ABC-DLBCL cells is oncogenic.^{8,9} Thus, for patients with a Cluster 3 phenotype, an attractive direction for future clinical trials is to test the efficacy of Jak/STAT3 inhibitors, such as those currently in clinical trials for myeloid proliferative diseases.³⁵ Recently, Lam et al have also shown that inhibition of both NF-κB and STAT3 pathways may be synergetic in enhancing tumor cell apoptosis in DLBCL cell lines with STAT3 activation⁹.

### Example B

### The 11 Gene Model

### Methods

Patients Information and Gene Expression Profiling Analysis:

The same as described in Example A.

Identification of Candidate Genes for Prediction of STAT3 Activation Status in DLBCL:

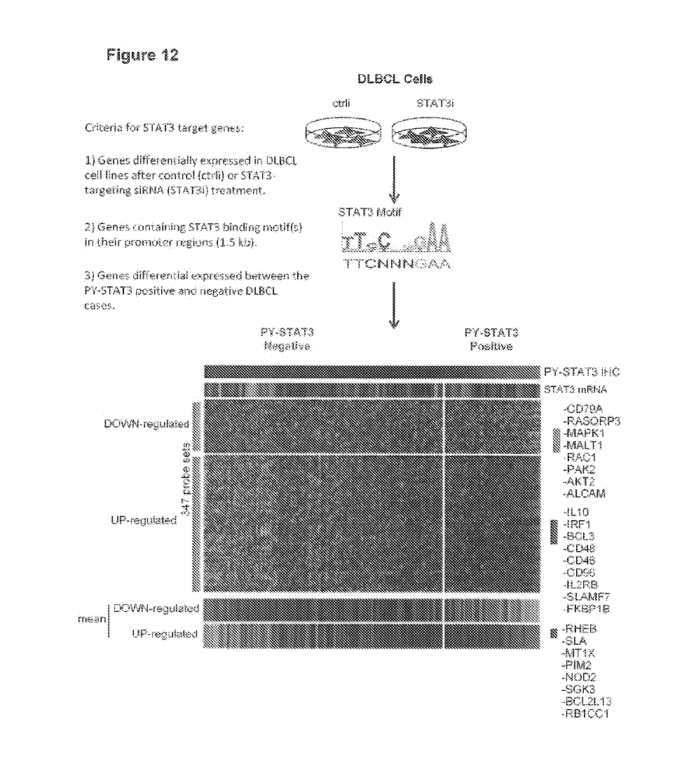

As illustrated in FIG. 12, a set of 265 candidate genes (347 probesets) was identified based on the following criteria: (i) differential gene expression based on the SAM²³ algorithm following STAT3 siRNA treatment in ABC-DLBCL cell lines; (ii) at least one STAT3 binding site can be recognized in the promoter region (1.5 kb sequence retrieved through the use of PAINT³⁶) by the FIMO³⁷ algorithm in combination with either the TRANSFAC³⁸ database or a computationally generated STAT3 site collection³⁹ (significance cut-off <=0.0001); (iii) differential gene expression between PY-STAT3-positive and -negative DLBCL tumors (t-test P<0.05, fold change >2).

Construction of the 11-Gene STAT3 Activation Signature for DLBCL Prognostication:

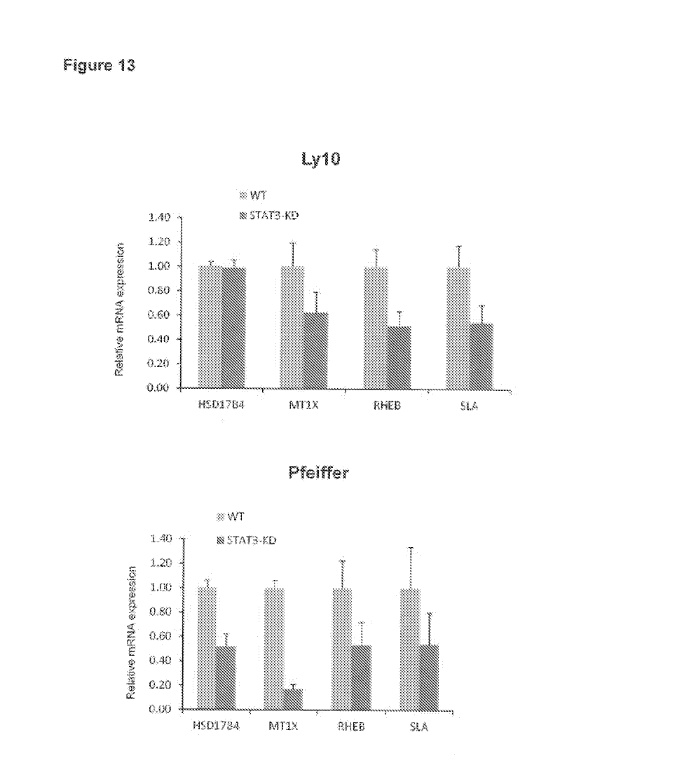

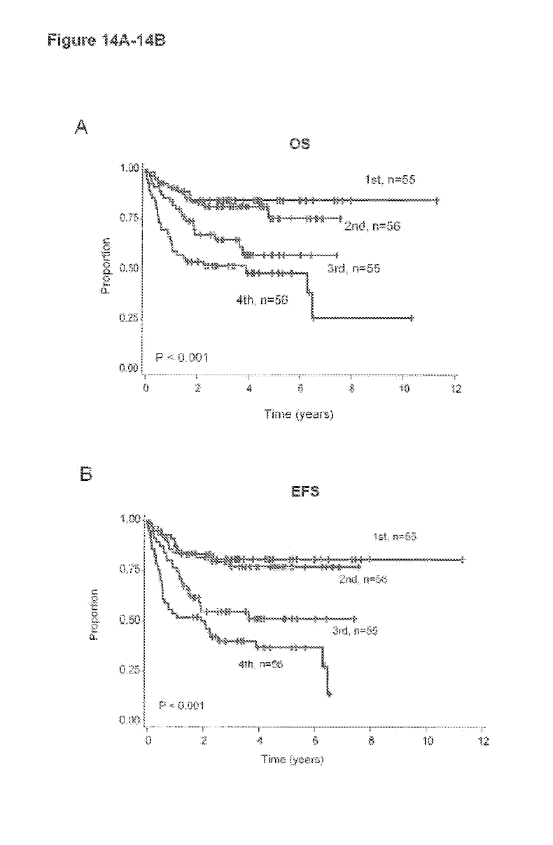

The above STAT3 target set was trained for prognostic prediction using the semi-supervisory (SSP) algorithm, with leave-one-out cross validation avoiding over-fitting.²⁴ Eleven probe-sets were selected from the 347-probe-set pool of STAT3 target genes by fitting the clinical outcome (OS) with the Cox proportional hazards model (P<0.05)²⁴, and comparing the consistency of their expression between the patients and cell line GEP data (Table 7). Four of these genes were validated by qRT-PCR in two ABC-DLBCL cell lines treated with STAT3 siRNA (FIG. 13).

### Results

Characteristics of the 11-Gene Signature:

As expected, known STAT3 target genes, such as CD48, CD96, IRF1, IL10, BCL3, and IL2RB were highly expressed in the PY-STAT3 positive tumors⁴⁰⁻⁴², whereas the PY-STAT3 negative tumors express high levels of RAC1, MAPK1 and AKT2 (FIG. 12). In addition, a previously reported gene signature for IL-10⁹, a STAT3-activating cytokine, was highly expressed in the PY-STAT3 positive cases (T-test, P=0.037; not shown).

The 11-Gene PY-STAT3 Signature Predicted Survival in DLBCL Patients Treated with R-CHOP and CHOP.

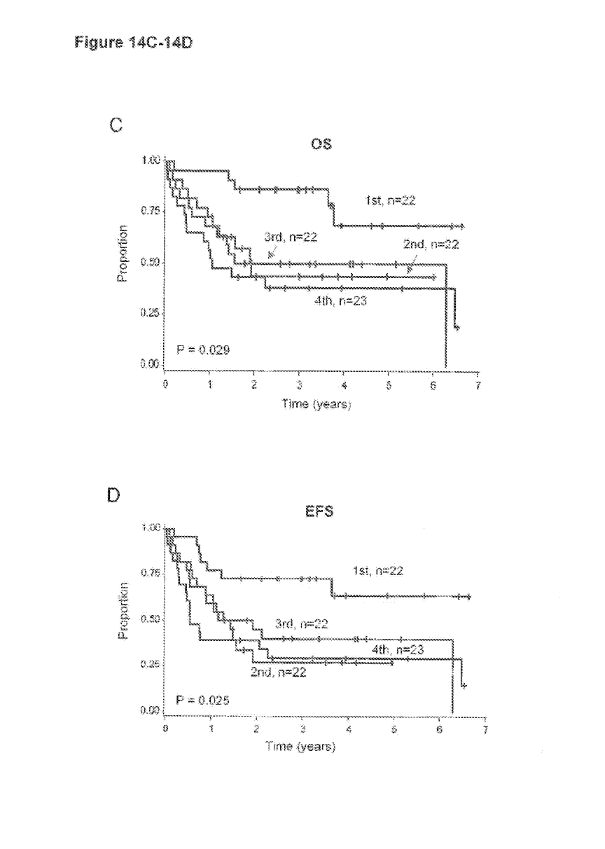

A previously published cohort of 222 DLBCL cases¹⁴ was divided into 4 quartiles using the average expression of this 11-gene predictor. To confirm that this quartile approach is biologically valid, the distribution of PY-STAT3 expression was examined in the quartile subgroups for a cohort of 98 cases for which both PY-STAT3 IHC score and GEP data were available. As shown in Table 8, the PY-STAT3 positive cases were significantly correlated with the expression of the STAT3 signature for the whole cohort (Chi-square test, P=0.001) as well as the ABC subgroup (Chi-square test, P=0.005). Most significantly, the PY-STAT3 signature separated the entire cohort of 222 patients into prognostically distinct quartile subgroups with 5-year OS rates of 84%, 81%, 57%, and 48%, and 5-year EFS rates of 81%, 77%, 51%, and 40% (P_{OS}<0.001; P_{EFS}<0.001, FIG. 14A-B). Similarly observation was made among the ABC cases in that the first quartile showed the most favorable outcome compared to the other three quartile subgroups (P_{OS}=0.029; P_{EFS}=0.025, FIG. 14C-D). These results demonstrate that the 11 gene PY-STAT3 signature effectively reports STAT3 activity in tumor cells and that this gene expression model can be used to predict survival in patients treated with R-CHOP. Applying this 11-gene PY-STAT3 signature, an association was also observed an with OS in a cohort of 181 DLBCL patient treated with CHOP (P=0.067, FIG. 15) and in another small cohort of 69 DLBCL cases treated with R-CHOP⁴³ (data not shown). The reduced predictive power among CHOP treated patients may be related to the R-CHOP-focused strategy that was used to derive this PY-STAT3 signature.

### Discussion

Additional insights into mechanism of resistance to the R-CHOP therapy may also be gleaned from the PY-STAT3 signature itself. Of the 11 genes in the signature, 6 have been studied functionally to various extents. HSD17B4 is a dehydrogenase involved in the peroxisomal fatty acid beta-oxidation. Its overexpression was recently reported to be a poor prognosticator in prostate cancer patients.⁴⁴ SLC2A13 encodes a H⁺-myo-inositol transporter that has been suggested to be a marker for cancer stem cells in an oral squamous cell carcinoma.⁴⁵ Aberrant expression of MT1X, which encodes metallothionein isoform 1, has been observed in several kinds of carcinomas, and its overexpression was correlated with enhanced drug resistance and shorter survival^{46,47} SLA encodes a Src-like adaptor protein (SLAP) that negatively regulates antigen-stimulated immune response.⁴⁸ It has not been implicated in lymphomas previously. RHEB is a key regulator in the PI3K/Akt/mTOR pathway that directly activates mTOR1 activity.⁴⁹ Cell type-specific oncogenic activity has been shown for RHEB especially in the context of PTEN haploinsufficiency.⁵° This is particular interesting in light of the previous report that PTEN loss occurs in 11% of GCB-DLBCL.⁵¹ Finally, ZNF420 encodes the KRAB-type zinc finger protein, Apak, which has been implicated in DNA damage and oncogene-induced stress response.⁵²

With the PY-STAT3-based gene signature model, strong associations were found with OS and EFS in a published cohort of 222 patients treated with R-CHOP. While the overall conclusion parallels the findings with PY-STAT3 IHC, this gene expression based model is amenable to future technologies such as diagnostic gene chips at the point-of-care. Prior to this report, two GEP-based DLBCL prognostic models have been reported by the LLMPP consortium, namely the bivariate GCB/ABC model^{5,6} and the trivariate model derived from the GCB, stromal-1 and stromal-2 GEP signatures¹⁴. Interestingly, although the current 11-gene signature is a much simpler univariate predictor, its survival predictive power is quite comparable to the trivariate model specifically constructed to incorporate tumor stromal contribution. One possible explanation for the advantage of the current model is the fact that STAT3 activation within the tumor cells is not only influenced by cell intrinsic genetic alterations, it also incorporates cytokine and growth factor cues in the tumor microenvironment. In other words, STAT3 activation is a holistic readout of the entire tumor tissue.

It is pertinent to point out here that Lam et al have previously classified a group of CHOP-treated ABC-DLBCL patients into STAT3-high and STAT3-low subgroups using a Lymphochip-derived GEP signature but did not observe prognostic differences between these two subgroups.⁹ In this regard, the 11-gene PY-STAT3 signature developed in the current study has at least three benefits compared to the signature used by Lam et al: 1) the current signature was cross-validated for correlation with PY-STAT3 expression in primary tumors; 2) direct STAT3 target genes are selected with the requirement of high affinity STAT3 binding site(s) in the promoter region; and most importantly, 3) the ability to predict survival among R-CHOP treated patients was used as a filtering criteria.

### REFERENCES

1. Anderson J R, Armitage J O, Weisenburger D D: Epidemiology of the non-Hodgkin's lymphomas: distributions of the major subtypes differ by geographic locations. Non-Hodgkin's Lymphoma Classification Project. Ann Oncol 9:717-20, 1998
2. Fisher R I, Gaynor E R, Dahlberg S, et al: Comparison of a standard regimen (CHOP) with three intensive chemotherapy regimens for advanced non-Hodgkin's lymphoma. N Engl J Med 328:1002-6, 1993
3. Coiffier B, Lepage E, Briere J, et al: CHOP chemotherapy plus rituximab compared with CHOP alone in elderly patients with diffuse large-B-cell lymphoma. N Engl J Med 346:235-42, 2002
4. Staudt L M, Dave S: The biology of human lymphoid malignancies revealed by gene expression profiling. Adv Immunol 87:163-208, 2005
5. Rosenwald A, Wright G, Chan W C, et al: The use of molecular profiling to predict survival after chemotherapy for diffuse large-B-cell lymphoma. N Engl J Med 346:1937-47, 2002
6. Alizadeh A A, Eisen M B, Davis R E, et al: Distinct types of diffuse large B-cell lymphoma identified by gene expression profiling. Nature 403:503-11, 2000
7. Davis R E, Brown K D, Siebenlist U, et al: Constitutive nuclear factor kappaB activity is required for survival of activated B cell-like diffuse large B cell lymphoma cells. J Exp Med 194:1861-74, 2001
8. Ding B B, Yu J J, Yu R Y, et al: Constitutively activated STAT3 promotes cell proliferation and survival in the activated B-cell subtype of diffuse large B-cell lymphomas. Blood 111:1515-23, 2008
9. Lam L T, Wright G, Davis R E, et al: Cooperative signaling through the signal transducer and activator of transcription 3 and nuclear factor-{kappa}B pathways in subtypes of diffuse large B-cell lymphoma. Blood 111:3701-13, 2008
10. Wright G, Tan B, Rosenwald A, et al: A gene expression-based method to diagnose clinically distinct subgroups of diffuse large B cell lymphoma. Proc Natl Acad Sci USA 100:9991-6, 2003
11. Pfreundschuh M, Ho A D, Cavallin-Stahl E, et al: Prognostic significance of maximum tumour (bulk) diameter in young patients with good-prognosis diffuse large-B-cell lymphoma treated with CHOP-like chemotherapy with or without rituximab: an exploratory analysis of the MabThera International Trial Group (MInT) study. Lancet Oncol 9:435-44, 2008
12. Sehn L H, Donaldson J, Chhanabhai M, et al: Introduction of combined CHOP plus rituximab therapy dramatically improved outcome of diffuse large B-cell lymphoma in British Columbia. J Clin Oncol 23:5027-33, 2005
13. Fu K, Weisenburger D D, Choi W W, et al: Addition of rituximab to standard chemotherapy improves the survival of both the germinal center B-cell-like and non-germinal center B-cell-like subtypes of diffuse large B-cell lymphoma. J Clin Oncol 26:4587-94, 2008
14. Lenz G, Wright G, Dave S S, et al: Stromal gene signatures in large-B-cell lymphomas. N Engl J Med 359:2313-23, 2008
15. Darnell J E: Validating Stat3 in cancer therapy. Nat Med 11:595-6, 2005
16. Yu H, Jove R: The STATs of cancer—new molecular targets come of age. Nat Rev Cancer 4:97-105, 2004
17. Yu H, Pardoll D, Jove R: STATs in cancer inflammation and immunity: a leading role for STAT3. Nat Rev Cancer 9:798-809, 2009
18. Nelson E A, Walker S R, Kepich A, et al: Nifuroxazide inhibits survival of multiple myeloma cells by directly inhibiting STAT3. Blood 112:5095-102, 2008
19. Burger R, Le Gouill S, Tai Y T, et al: Janus kinase inhibitor INCB20 has antiproliferative and apoptotic effects on human myeloma cells in vitro and in vivo. Mol Cancer Ther 8:26-35, 2009
20. Kube D, Holtick U, Vockerodt M, et al: STAT3 is constitutively activated in Hodgkin cell lines. Blood 98:762-70, 2001
21. Chiarle R, Simmons W J, Cai H, et al: Stat3 is required for ALK-mediated lymphomagenesis and provides a possible therapeutic target. Nat Med 11:623-9, 2005
22. Hans C P, Weisenburger D D, Greiner T C, et al: Confirmation of the molecular classification of diffuse large B-cell lymphoma by immunohistochemistry using a tissue microarray. Blood 103:275-82, 2004
23. Tusher V G, Tibshirani R, Chu G: Significance analysis of microarrays applied to the ionizing radiation response. Proc Natl Acad Sci USA 98:5116-21, 2001
24. Bair E, Tibshirani R: Semi-supervised methods to predict patient survival from gene expression data. PLoS Biol 2:E108, 2004
25. Simon R, Peng A: BRB-ArrayTools User Guide, version 4.2.0-Beta_{—}1. Biometric Research Branch, National Cancer Institute., 2011
26. Rosenwald A, Wright G, Wiestner A, et al: The proliferation gene expression signature is a quantitative integrator of oncogenic events that predicts survival in mantle cell lymphoma. Cancer Cell 3:185-97, 2003
27. Moreaux J, Cremer F W, Reme T, et al: The level of TACI gene expression in myeloma cells is associated with a signature of microenvironment dependence versus a plasmablastic signature. Blood 106:1021-30, 2005
28. Cattoretti G, Shaknovich R, Smith P M, et al: Stages of germinal center transit are defined by B cell transcription factor coexpression and relative abundance. J Immunol 177:6930-9, 2006
29. Saito M, Gao J, Basso K, et al: A signaling pathway mediating downregulation of BCL6 in germinal center B cells is blocked by BCL6 gene alterations in B cell lymphoma. Cancer Cell 12:280-92, 2007
30. Kwon H, Thierry-Mieg D, Thierry-Mieg J, et al: Analysis of interleukin-21-induced Prdm1 gene regulation reveals functional cooperation of STAT3 and IRF4 transcription factors. Immunity 31:941-52, 2009
31. Cenci S, Mezghrani A, Cascio P, et al: Progressively impaired proteasomal capacity during terminal plasma cell differentiation. EMBO J 25:1104-13, 2006
32. Shah J J, Orlowski R Z: Proteasome inhibitors in the treatment of multiple myeloma. Leukemia 23:1964-79, 2009
33. Dunleavy K, Pittaluga S, Czuczman M S, et al: Differential efficacy of bortezomib plus chemotherapy within molecular subtypes of diffuse large B-cell lymphoma. Blood 113:6069-76, 2009
34. Leonard J P, Furman R R, Cheung Y K, et al: CHOP-R+bortezomib as initial therapy for diffuse large B-cell lymphoma (DLBCL). Proc Am Soc Clin Oncol 25, 2007
35. Verstovsek S: Therapeutic potential of JAK2 inhibitors. Hematology Am Soc Hematol Educ Program: 636-42, 2009
36. Vadigepalli R, Chakravarthula P, Zak D E, et al: PAINT: a promoter analysis and interaction network generation tool for gene regulatory network identification. OMICS 7:235-52, 2003
37. Bailey T L, Boden M, Buske F A, et al: MEME SUITE: tools for motif discovery and searching. Nucleic Acids Res 37:W202-8, 2009
38. Wingender E, Chen X, Hehl R, et al: TRANSFAC: an integrated system for gene expression regulation. Nucleic Acids Res 28:316-9, 2000
39. Vallania F, Schiavone D, Dewilde S, et al: Genome-wide discovery of functional transcription factor binding sites by comparative genomics: the case of Stat3. Proc Natl Acad Sci USA 106:5117-22, 2009
40. Lam L T, Davis R E, Ngo V N, et al: Compensatory IKKalpha activation of classical NF-kappaB signaling during IKKbeta inhibition identified by an RNA interference sensitization screen. Proc Natl Acad Sci USA 105:20798-803, 2008
41. Brocke-Heidrich K, Ge B, Cvijic H, et al: BCL3 is induced by IL-6 via Stat3 binding to intronic enhancer HS4 and represses its own transcription. Oncogene 25:7297-304, 2006
42. Takeda T, Kurachi H, Yamamoto T, et al: Crosstalk between the interleukin-6 (IL-6)-JAK-STAT and the glucocorticoid-nuclear receptor pathway: synergistic activation of IL-6 response element by IL-6 and glucocorticoid. J Endocrinol 159:323-30, 1998
43. Shaknovich R, Geng H, Johnson NA, et al: DNA methylation signatures define molecular subtypes of diffuse large B-cell lymphoma. Blood 116:e81-9, 2010
44. Rasiah K K, Gardiner-Garden M, Padilla E J, et al: HSD17B4 overexpression, an independent biomarker of poor patient outcome in prostate cancer. Mol Cell Endocrinol 301:89-96, 2009
45. Lee D G, Lee J H, Choi B K, et al: H(+)-myo-inositol transporter SLC2A13 as a potential marker for cancer stem cells in an oral squamous cell carcinoma. Curr Cancer Drug Targets 11:966-75, 2011
46. Arriaga J M, Levy E M, Bravo A I, et al: Metallothionein expression in colorectal cancer: relevance of different isoforms for tumor progression and patient survival. Hum Pathol 43:197-208, 2012
47. Chun J H, Kim H K, Kim E, et al: Increased expression of metallothionein is associated with irinotecan resistance in gastric cancer. Cancer Res 64:4703-6, 2004
48. Park S K, Qiao H, Beaven M A: Src-like adaptor protein (SLAP) is upregulated in antigen-stimulated mast cells and acts as a negative regulator. Mol Immunol 46:2133-9, 2009
49. Janakiram M, Thirukonda V K, Sullivan M, et al: Emerging Therapeutic Targets in Diffuse Large B-Cell Lymphoma. Curr Treat Options Oncol, 2012
50. Nardella C, Chen Z, Salmena L, et al: Aberrant Rheb-mediated mTORC1 activation and Pten haploinsufficiency are cooperative oncogenic events. Genes Dev 22:2172-7, 2008
51. Lenz G, Wright G W, Emre N C, et al: Molecular subtypes of diffuse large B-cell lymphoma arise by distinct genetic pathways. Proc Natl Acad Sci USA 105:13520-5, 2008
52. Wang S, Tian C, Xing G, et al: ARF-dependent regulation of ATM and p53 associated KZNF (Apak) protein activity in response to oncogenic stress. FEBS Lett 584:3909-15, 2010

1. A method of classifying a human patient with diffuse large B-cell lymphoma (DLBCL), the method comprising determining mRNA expression levels of human genes in a DLBCL biopsy specimen from the patient, wherein the genes comprise HSD17B4, RNF149, ZNF805, SLC2A13, RHEB, MT1X, NAT8L, C15orf29, ZNF420, PCNX and SLA, so as to classify the DLBCL patient based on expression levels.

2. The method of claim 1, wherein the genes for which expression is determined are predictive of activation of signal transducer and activator of transcription 3 (STAT3).

3. The method of claim 1, wherein the patient is classified into a subgroup by comparing the expression of genes from the patient with the expression of the same genes from a cohort of DLBCL patients who have already been classified into subgroups.

4. The method of claim 1, wherein patients are classified into one of four quartile subgroups.

5. The method of claim 1, wherein a patient classified into the bottom 50% subgroup has a more favorable outcome of survival compared to patients in the top gene expression quartile.

6. The method of claim 1, wherein for a patient in the non-GCB/ABC subgroup, a patient classified into the bottom gene expression quartile has a more favorable survival outcome compared to patients in the other quartile subgroups.

7. A microarray for classifying a human patient with diffuse large B-cell lymphoma (DLBCL), where the microarray comprises nucleic acid probes for genes HSD17B4, RNF149, ZNF805, SLC2A13, RHEB, MT1X, NAT8L, C15orf29, ZNF420, PCNX and SLA.

8. A method of classifying a human patient with diffuse large B-cell lymphoma (DLBCL), the method comprising
determining mRNA expression levels of human genes in a DLBCL biopsy specimen from the patient, wherein the genes comprise Module A genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2 and ZNRF1, and Module B genes BTLA, C13orf18, CFLAR, EV12A, HIST2H2AA3, IL16, IL2RA and PTGER4; and
determining the expression levels of Module A genes and the expression levels of Module B genes so as to classify the DLBCL patient based on the expression levels.

9. The method of claim 8, wherein the genes for which expression is determined are predictive of activation of signal transducer and activator of transcription 3 (STAT3).

10. The method of claim 8, wherein the patient is classified into one of four clusters by comparing the expression of Module A and Module B genes from the patient with the expression of Module A and Module B genes from a cohort of DLBCL patients who have already been classified into one of the four clusters.

11. The method of claim 8, wherein the patient is classified in Cluster 1 if the majority of genes in Module A is downregulated and if the majority of genes in Module B is down-regulated.

12. The method of claim 8, wherein the patient is classified in Cluster 2 if the majority of genes in Module A is upregulated and if the majority of genes in Module B is not upregulated.

13. The method of claim 8, wherein the patient is classified in Cluster 3 if the majority of genes in Module A is upregulated and if the majority of genes in Module B is upregulated.

14. The method of claim 8, wherein the patient is classified in Cluster 4 if the majority of genes in Module A is not upregulated and if the majority of genes in Module B is upregulated.

15. The method of claim 8,
wherein the patient is classified in Cluster 1 if the majority of genes in Module A is downregulated and if the majority of genes in Module B is down-regulated;
wherein the patient is classified in Cluster 2 if the majority of genes in Module A is upregulated and if the majority of genes in Module B is not upregulated;
wherein the patient is classified in Cluster 3 if the majority of genes in Module A is upregulated and if the majority of genes in Module B is upregulated; and
wherein the patient is classified in Cluster 4 if the majority of genes in Module A is not upregulated and if the majority of genes in Module B is upregulated.

16. The method of claim 8, wherein the patient is classified into one of four clusters by determining y_{pred} for Module A and y_{pred} for Module B, where y_{pred}=b₀+b₁x₁+b₂x₂+ . . . +bₙxₙ, where x₁, x₂ . . . xₙ is the expression value of each gene, and where the coefficients b₀, b₁ . . . +bₙ are set forth in Table 5;
wherein the patient is classified in Cluster 1 if y_{pred} for Module A and y_{pred} for Module B are both negative;
wherein the patient is classified in Cluster 2 if y_{pred} for Module A is positive and if y_{pred} for Module B is negative;
wherein the patient is classified in Cluster 3 if y_{pred} for Module A and y_{pred} for Module B are both positive; and
wherein the patient is classified in Cluster 4 if y_{pred} for Module A is negative and if y_{pred} for Module B is positive.

17. The method of claim 8, wherein a patient classified in Cluster 4 is predicted to be the least likely to benefit from therapy with rituximab in combination with cyclophosphamide, doxorubicin, vincristine, and prednisone (R-CHOP), compared to a patient in Cluster 1, 2 or 3.

18. The method of claim 8, wherein a DLBCL patient undergoing therapy with a combination of cyclophosphamide, doxorubicin, vincristine, and prednisone (CHOP) classified in Cluster 2 is predicted to have a more favorable likelihood of survival compared to a patient classified in Cluster 3.

19. The method of claim 8, wherein a patient classified in Cluster 1 or 3 is predicted to benefit the most from therapy with rituximab in combination with cyclophosphamide, doxorubicin, vincristine, and prednisone (R-CHOP), compared to a patient in Cluster 2 or 4.

20. A method of determining the prognosis of a diffuse large B-cell lymphoma (DLBCL) patient undergoing treatment with rituximab in combination with cyclophosphamide, doxorubicin, vincristine, and prednisone (R-CHOP), or treatment with rituximab in combination with cyclophosphamide, mitoxantrone, vincristine, and prednisone (R-CNOP), the method comprising determining the level of phospho-Tyr705-STAT3 (PY-STAT3) in a DLBCL biopsy specimen from the patient using immunohistochemistry, wherein PY-STAT3 positivity predicts a poor likelihood of survival in comparison to a patient with PY-STAT3 negativity.

21. The method of claim 20, wherein PY-STAT3 positivity or negativity is determined by scoring the intensity of PY-STAT3 staining using a 4-tiered scale (0, 3, 6, 9), scoring the percentage of PY-STAT3 stained DLBCL tumor cells using a 10-tiered scale (0-9), and multiplying the two scores together to obtain a case score for the patient, where a case score with a value of 15 or greater is considered positive and a case score with a value below 15 is considered negative.

22. The method of claim 20, wherein the patient is a non-germinal center B-cell-like (non-GCB) DLBCL patient.

23. A method of determining the prognosis of a diffuse large B-cell lymphoma (DLBCL) patient undergoing treatment with a combination of cyclophosphamide, doxorubicin, vincristine, and prednisone (CHOP), or with a combination of cyclophosphamide, mitoxantrone, vincristine, and prednisone (CNOP), the method comprising determining the level of phospho-Tyr705-STAT3 (PY-STAT3) and the level of BCL6 in a DLBCL biopsy specimen from the patient using immunohistochemistry, wherein PY-STAT3 positivity and BCL6 negativity predicts a poor likelihood of survival in comparison to a patient who is not PY-STAT3 positive and BCL6 negative.

24. The method of claim 23, wherein PY-STAT3 positivity or negativity is determined by scoring the intensity of PY-STAT3 staining using a 4-tiered scale (0, 3, 6, 9), scoring the percentage of PY-STAT3 stained DLBCL tumor cells using a 10-tiered scale (0-9), and multiplying the two scores together to obtain a case score for the patient, where a case score with a value of 15 or greater is considered positive and a case score with a value below 15 is considered negative, and wherein the patient is considered BCL6 positive if 30% or more of the DLBCL tumor cells stain positive for BCL6, and BCL6 negative if less than 30% of the DLBCL tumor cells stain positive for BCL6.

25. A microarray for classifying a human patient with diffuse large B-cell lymphoma (DLBCL), where the microarray comprises nucleic acid probes for genes MEX3D, BATF, CAPN2, CCND2, CD2, CMTM3, DYNLT1, ELL2, GALNT1, GCA, GMFG, GYG1, GZMB, MAN1A1, MT1X, PERP, PLAGL1, PRF1, RAB27A, S100A6, SERPINB1, TTC39C, XK, ZBED2, ZNRF1, BTLA, C13orf18, CFLAR, EV12A, HIST2H2AA3, IL16, IL2RA and PTGER4.

26. A method of classifying a human patient with diffuse large B-cell lymphoma (DLBCL), the method comprising
determining STAT3 mRNA expression level in a DLBCL biopsy specimen from the patient, and
comparing the level of STAT3 mRNA expression from the patient with the level of expression of STAT3 mRNA from a cohort of DLBCL patients,
wherein a patient with a level of STAT3 mRNA expression that is greater than 1 standard deviation above the mean level of STAT3 mRNA expression in the cohort has a less favorable survival outcome compared to patients having a level of STAT3 mRNA expression that is less than 1 standard deviation below the mean level of STAT3 mRNA expression in the cohort.
